SEQUENCE LISTING (42 sequences)

SEQ ID NO: 1 - DNA, 24 bp, Artificial Sequence
  primer
atcctggatactcacgacgcggac

SEQ ID NO: 2 - DNA, 25 bp, Artificial Sequence
  primer
catcaacctctcatggcaagaattt

SEQ ID NO: 3 - DNA, 27 bp, Artificial Sequence
  primer
aggtgaatggctctgaaaatttgtctc

SEQ ID NO: 4 - DNA, 30 bp, Artificial Sequence
  primer
agagtttaattgtaatgctgttttgacaca

SEQ ID NO: 5 - DNA, 20 bp, Artificial Sequence
  primer
cagcacgaagatcactggaa

SEQ ID NO: 6 - DNA, 20 bp, Artificial Sequence
  primer
tgaggaaaaggagcagagga

SEQ ID NO: 7 - DNA, 24 bp, Artificial Sequence
  primer
ctgctgctccttgaggcatccaca

SEQ ID NO: 8 - DNA, 26 bp, Artificial Sequence
  primer
cttctggctgttccagtactcggcat

SEQ ID NO: 9 - DNA, 24 bp, Artificial Sequence
  primer
ctgctactccttgaggcatccaca

SEQ ID NO: 10 - DNA, 26 bp, Artificial Sequence
  primer
cttctggctgttccaggactcggcga

SEQ ID NO: 11 - DNA, 24 bp, Artificial Sequence
  primer
ctgctgctccctgaggcatccaca

SEQ ID NO: 12 - DNA, 26 bp, Artificial Sequence
  primer
cttctggctgttccagtactcagcgt

SEQ ID NO: 13 - DNA, 26 bp, Artificial Sequence
  primer
cttctggctgttccagtactcctcat

SEQ ID NO: 14 - DNA, 26 bp, Artificial Sequence
  primer
cttctggctgttccagtgctccgcag

SEQ ID NO: 15 - DNA, 26 bp, Artificial Sequence
  primer
cttctggctgttccagtactcggcgc

SEQ ID NO: 16 - DNA, 26 bp, Artificial Sequence
  primer
gcacgtttcttgtggcagcttaagtt

SEQ ID NO: 17 - DNA, 21 bp, Artificial Sequence
  primer
atgcacgggaggccatacggt

SEQ ID NO: 18 - DNA, 26 bp, Artificial Sequence
  primer
gcacgtttcttgtggcagctaaagtt

SEQ ID NO: 19 - DNA, 21 bp, Artificial Sequence
  primer
atgcacaggaggccatagggt

SEQ ID NO: 20 - DNA, 22 bp, Artificial Sequence
  primer
tttcctgtggcagcctaagagg

SEQ ID NO: 21 - DNA, 21 bp, Artificial Sequence
  primer
atgcatgggaggcaggaagca

SEQ ID NO: 22 - DNA, 24 bp, Artificial Sequence
  primer
cacagcacgtttcttggagtactc

SEQ ID NO: 23 - DNA, 24 bp, Artificial Sequence
  primer
cagatgcatgggaggcaggaagcg

SEQ ID NO: 24 - DNA, 27 bp, Artificial Sequence
  primer
agcacgtttcttggagcaggttaaaca

SEQ ID NO: 25 - DNA, 21 bp, Artificial Sequence
  primer
atgcatgggaggcaggaagcg

SEQ ID NO: 26 - DNA, 24 bp, Artificial Sequence
  primer
cacagcacgtttcctgtggcaggg

SEQ ID NO: 27 - DNA, 33 bp, Artificial Sequence
  primer
tggaatgtctaaagcaagctatttaacatatgt

SEQ ID NO: 28 - DNA, 24 bp, Artificial Sequence
  primer
cacagcacgtttcttgaagcagga

SEQ ID NO: 29 - DNA, 23 bp, Artificial Sequence
  primer
acagcacgtttcttggaggaggt

SEQ ID NO: 30 - DNA, 20 bp, Artificial Sequence
  primer
gccagggagggaaatcaact

SEQ ID NO: 31 - DNA, 21 bp, Artificial Sequence
  primer
atccagtggaggacacagcac

SEQ ID NO: 32 - DNA, 24 bp, Artificial Sequence
  primer
aagaaacaaactgccccttacacc

SEQ ID NO: 33 - DNA, 26 bp, Artificial Sequence
  primer
tagtattgcccctagtcactgtcaag

SEQ ID NO: 34 - DNA, 24 bp, Artificial Sequence
  primer
aagaaacaaactgccccttatacc

SEQ ID NO: 35 - DNA, 26 bp, Artificial Sequence
  primer
tagtactgcccctagtcactgccaag

SEQ ID NO: 36 - DNA, 26 bp, Artificial Sequence
  primer
tagtactgtccctagtcactgccaag

SEQ ID NO: 37 - DNA, 24 bp, Artificial Sequence
  primer
ctctcttgaccacgctggtaccta

SEQ ID NO: 38 - DNA, 25 bp, Artificial Sequence
  primer
ttggcctcttggctatacctctttt

SEQ ID NO: 39 - DNA, 24 bp, Artificial Sequence
  primer
cctcctgaccctgatgacagtcct

SEQ ID NO: 40 - DNA, 24 bp, Artificial Sequence
  primer
ccatctgcccctcaagcacctcaa

SEQ ID NO: 41 - DNA, 26 bp, Artificial Sequence
  primer
ctcagtgctcgcccctccctagtgat

SEQ ID NO: 42 - DNA, 26 bp, Artificial Sequence
  primer
ctcagtgctcgcccctccctagtgat

(19) United States
(12) Patent Application Publication
(10) Pub. No.: US 2024/0060129 A1
(43) Pub. Date: Feb. 22, 2024

(54) METHODS, COMPOSITIONS AND KITS FOR HLA TYPING

(71) Applicants: THE UNIVERSITY OF BIRMINGHAM, Birmingham West Midlands (GB); UNIVERSITY HOSPITAL BIRMINGHAM NHS FOUNDATION TRUST, Birmingham West Midlands (GB)

(72) Inventors: Thomas George Nieto, Birmingham West Midlands (GB); Joanne Dawn Stockton, Birmingham West Midlands (GB); Andrew David Beggs, Birmingham West Midlands (GB)

(21) Appl. No.: 17/914,759

(22) PCT Filed: Mar. 26, 2021
(86) PCT No.: PCT/GB2021/050757

§ 371 Date: Sep. 26, 2022

(30) Foreign Application Priority Data
Mar. 27, 2020 (GB) 2004528.2

(51) Int. Cl. C12Q 1/6881

(52) U.S. Cl. CPC C12Q 1/6881; C12Q 2600/16

(57) ABSTRACT

The invention relates to a set of oligonucleotides, and a kit comprising a set of oligonucleotides where the oligonucleotides are for use in determining the HLA genotype of a DNA sample. The invention also relates to a method of determining the HLA genotype of a DNA sample. The method may be used to identify a suitable donor and/or recipient of a transplant, for paternity testing, to identify the HLA type for determination of epitope binding capability in neo-antigen prediction, or for diagnosing an immune disorder such as ankylosing spondylitis. The method preferably uses long-range PCR and long read sequencing preferably using a Type R10 nanopore.

### FIELD OF THE INVENTION

The present invention relates to methods, compositions and kits for performing high-resolution HLA typing and phasing.

### BACKGROUND OF THE INVENTION

Modern organ transplantation techniques (1) have only been made possible by the development of potent immunosuppressive agents (2) and the identification of the Human Leucocyte Antigen/Major Histocompatibility Complex (3) as the determinant of recognition of a transplanted organ as “foreign”.

Transplant rejection is a substantial challenge in solid organ transplantation, whilst Graft versus Host Disease (GVHD) is a common complication following an allogenic tissue transplant, such as a stem cell or bone marrow transplant. Rejection of the transplant may be mediated by both T cells and B cells and can lead to significant complications in organ function or failure.

Preservation of organ viability prior to and during the implantation procedure is a second significant challenge. The removal, storage and transplantation of an organ may profoundly affect the internal structure and function of the organ and can influence significantly the degree to which the return of normal organ function is delayed or prevented after transplantation is completed. The time period in which solid human organs may be effectively preserved varies by organ, with kidneys ranging from 24-36 hours, pancreas from 12-18 hours, liver from 8-12 hour and heart and lung from 4-6 hours.

The suitability of a transplant replies on matching a suitable donor and recipient, by ‘typing’ their human leukocyte antigen (HLA) alleles. The HLA system, found on the short arm of chromosome 6, is one of the most polymorphic regions of the human genome, encodes the Major Histocompatibility Complex (MHC) proteins and is responsible for regulating the adaptive immune system.

All nucleated cells in the human body expresses Class-I HLA genes (HLA-A, -B, and -C) and immune cells express some of the Class-II HLA genes (such as HLA-DRB1, -DQB1, etc.). These proteins are expressed on the cell surface and are responsible for antigen presentation and immunological memory mechanisms.

The HLA genes are co-dominant, both alleles on the two chromosomes are expressed, and are exceptionally polymorphic in the exons which are involved in antigen recognition.

To date, well over 15,000 HLA Class I and II alleles have been identified in the world population, with considerable variation observed across the entire HLA region. A single HLA molecule can display a range of immunogenic epitopes (variously recognised by T cells and by antibodies) with each determined by a specific, short series of base sequences of DNA and it is the linked combination of these specific sequences that defines each HLA allele.

The region of the HLA proteins that in turn vary in structure are those that interact with fragments of the pathogens (antigen presentation) and with immune receptors on T cells, B cells, and natural killer cells. This also renders HLA molecules highly immunogenic between individuals, leading for example to rejection in transplant situations.

Each HLA gene also comprises a linear series of introns and up to eight exons. The polymorphic regions are mostly within exons two and three for Class I HLA and exon two for Class II HLA, but not exclusively. Variation in other parts of the genes are also associated with expression variations (low or high) or null alleles (no protein product), and this includes the 3′ untranslated region. Low expression HLA variants are associated with better outcomes in HLA mismatched bone marrow transplantation and HLA antibody incompatible organ transplantation. Therefore, sequences determining both structural variants and expression variants are of clinical significance.

The nomenclature of the HLA region is necessarily complex, in order to allow a standardised reporting system between laboratories (5). This nomenclature is known as the WHO Nomenclature Committee for Factors of the HLA System, which starts with the name of the locus (i.e. HLA-A) followed by up to four fields indicating different levels of variation in the DNA sequence and the resulting protein. The first field defines a group of alleles that corresponds to the serologically defined specificity of HLA. The second field equates to non-synonymous base pair changes that lead to a change in the protein sequence and the third field demonstrates synonymous base pair changes that do not cause protein changes. The fourth field represents changes in the non-coding (i.e. intronic) regions.

During the process of organ transplantation, HLA typing is performed in order to determine suitability for transplant. The HLA genetics system uses an international classification standard based on observed allelic variation and a common system of representation on genes that make up the HLA region contiguously within chromosome 6 (HLA-A,B,C, DQA1, DPB1, DRB1/3/4/5 and others).

Kidney, pancreas, heart and liver transplantation rely on at least a two field match (6), whereas the ideal with allogenic stem cell transplantation would be a four field match (7) and currently the predominant technique used for this is either Sanger sequencing that provides second field resolution (8) and Sequence Specific PCR (SS-PCR) (9) for first field resolution, which uses groups of primers to span specific loci in the HLA regions. Although relatively quick (2 hours) this technique is limited by poor resolution to the first or second field only and requires the use of a dedicated real time PCR instrument.

The DNA-based methods currently used for clinical HLA testing involve rebuilding the likely starting sequence by combinations of multiple overlapping short sequences and statistical likelihood to determine the phasing of the separate sequences. Each of these sequence reads is typically shorter than each exon. Linking all polymorphic regions, and therefore defining the allele, is dependent on highly complex chemistry and procedures and is subject to phasing errors because of regions of homology and shared polymorphisms between related, but not identical, alleles. Thus, short reads preclude effective analysis of the haplotype and phasing of the HLA region, causing problems with accurate classifications of part of the HLA region, including regions with runs of homozygosity (11). Primer design around these regions using short read technology is challenging as variation makes it difficult to design primers that span anything other than very short regions, targeting specific alleles, The polymorphy of the HLA region, together with the high homology of these loci, makes the classical NGS (next generation sequencing) pipelines impractical: it is not the individual SNPs or indels, but whole exon or whole gene sequences identifying alleles that must be elucidated by NGS-based HLA typing. Further, use of this technology remains expensive, with a large capital outlay required for the sequencing instrument as well as the use of proprietary software. Short read technology is comparatively slow compared to SS-PCR as the library preparation and NGS steps takes greater than 24 hours, meaning that accurate four field deceased donor typing is a near impossibility.

Furthermore, as sequence based typing (SBT) focuses primarily on the previously mentioned important exons, the phasing problem known from whole-genome assembly can be the main source of ambiguity. During phasing the individual base differences are assigned unambiguously to one of the chromosomes. This cis/trans phase problem prevalent in HLA typing is not easily resolved when using short read technology; calculating the phase is hindered by sequencing artefacts, missing references, and other factors detailed below. These factors can introduce new typing issues different from phase ambiguity. Phase resolution can only rarely be resolved by use of a large number of short reads. Other issues with short read technology is the inability to find novel sequences or known alleles with unknown intronic parts; most of the novelties are in introns/UTRs, and these regions are not investigated as thoroughly as exons, as discussed above.

Therefore, there is a great need to develop new NGS-based HLA-typing strategies that can decipher the entire HLA loci of a subject, and which are accurate, faster, and more cost effective than current short read technologies, and which can routinely be used in a clinical laboratory. One difficulty is designing suitable primers to be able to perform such long-reads accurately across the HLA region.

Thus, in an aspect, there is provided a set of oligonucleotides comprising oligonucleotides of SEQ ID NOs: 1-11, 16-35 and 37-42 or variants thereof. In an embodiment, the set of oligonucleotides may further comprise one or more of oligonucleotides of SEQ ID NOs: 12, 13, 14, 15 and 36 or variants thereof.

In an embodiment, the set of oligonucleotides comprises oligonucleotides of SEQ ID NOs: 1-42.

The term “oligonucleotide” herein may be used interchangeably with the term “primer”.

As used herein, “HLA Class I oligonucleotides” refers to those oligonucleotides of SEQ ID NOs: 1-6 or variants thereof.

As used herein, “HLA Class II oligonucleotides” refers to those oligonucleotides of SEQ ID NOs: 7-42 or variants thereof.

Variants thereof may include one or more oligonucleotides of at least 95% sequence identity (such as 95%, such as 96%, such as 97%, such as 98%, such as 99% or more sequence identity) to an oligonucleotide of SEQ ID NOs: 142. Variants thereof may include one or more oligonucleotides corresponding to SEQ ID NOs: 1-11, 16-35 or 37-42 in which between 1 and 5 nucleotides (such as 1 nucleotide, such as 2 nucleotides, such as 3 nucleotides, such as 4 nucleotides, such as 5 nucleotides), are truncated from the 5′ and/or 3′ end of said oligonucleotide(s). Features giving rise to such variants are referred to as “variations”.

For example, in an embodiment, the set of oligonucleotides may comprise oligonucleotides of SEQ ID NOs: 1-11, oligonucleotides of at least 95% sequence identity to oligonucleotides of SEQ ID NOs: 16-35 and oligonucleotides corresponding to SEQ ID NOs: 37-42 in which between 1 and 5 nucleotides are truncated from the 5′ and/or 3′ end of said oligonucleotides (“truncations”). For example, the set of oligonucleotides may comprise oligonucleotides of SEQ ID NOs: 1-11, oligonucleotides of 95% sequence identity to oligonucleotides of SEQ ID NOs: 16-30, oligonucleotides of 98% sequence identity to oligonucleotides of SEQ ID NOs: 31-35, oligonucleotides corresponding to SEQ ID NOs: 37-40 in which 2 nucleotides are truncated from the 5′ and/or 3′ end of said oligonucleotides and oligonucleotides corresponding to SEQ ID NOs: 41-42 in which 4 nucleotides are truncated from the 5′ and/or 3′ end of said oligonucleotides. Therefore, the set of oligonucleotides may comprise any one of the variations of a given SEQ ID NO described above. The skilled person will appreciate that this is intended to exemplify how a set of oligonucleotides may vary, and is non-limiting.

In another aspect, there is provided a kit comprising the set of oligonucleotides of the first aspect. The kit may comprise oligonucleotides of SEQ ID NOs: 1-11, 16-35 and 37-42 or variants thereof. The set of oligonucleotides may further comprise one or more of oligonucleotides of SEQ ID NOs: 12, 13, 14, 15 and 36 or variants thereof. The set of oligonucleotides may comprise oligonucleotides of SEQ ID NOs: 1-42 or variants thereof.

The kit may also comprise one or more of, or all of, a set of instructions, a DNA amplification mix, and nuclease free water. The kit may also comprise one or more of, or all of, a barcoding mix, a ligation mix, an end repairing mix, a tailing mix, a clean-up mix, an adaptor mix, and an elution buffer.

A DNA amplification mix may comprise a DNA polymerase such as a Taq polymerase, dNTPs, and optionally comprising a DNA polymerase with 3′→5′ exonuclease activity. Preferably the DNA polymerase is a high-fidelity DNA polymerase, i.e. with an error rate of less than 10⁻⁵, such as less than 10⁻⁶.

The oligonucleotides may be provided lyophilised in an amount to be reconstituted in a suitable buffer, or the oligonucleotides may be provided in solution in a suitable buffer. The skilled person will be able to identify a suitable buffer which may be, for example, a Tris-EDTA (TE) buffer at around pH8.0 or nuclease free water,

The HLA Class I and HLA Class II oligonucleotides may each be provided separately. The HLA Class I and HLA Class II oligonucleotides may be provided together as a single mixture. Two or more of the HLA Class I and HLA Class II oligonucleotides may be provided together, with the remainder of the HLA Class I and HLA Class II oligonucleotides being provided in one or more further preparations. The HLA Class I oligonucleotides may be provided together. The HLA Class II oligonucleotides may be provided together. The oligonucleotides may be provided lyophilised or in a suitable buffer.

The set of oligonucleotides or the kit of any of the above aspects may be for use in determining the HLA genotype (herein referred to as “HLA typing”) of a DNA sample. The kit may be for use in performing a method of the invention.

In another aspect, there is provided a method of determining the HLA genotype (“HLA typing”) of a DNA sample comprising:
a) contacting the oligonucleotides or variants thereof according to the first aspect of the invention with the DNA sample and a DNA amplification mix (together referred to as the “amplification reaction mix”);
b) amplifying target sequences in the DNA sample using a primer-dependent DNA amplification method, such as PCR, thereby producing amplicons; and
c) determining the sequence of said amplicons.

Step a) and step b) of the method may be performed independently for a set of HLA Class I oligonucleotides, and for a set of HLA Class II oligonucleotides. The amplification products (amplicons) of step a) and step b) may be combined for step c).

In step b), the HLA Class I oligonucleotides may be provided at a concentration of about 20-200 μM, suitably about 50-150 μM, most suitably about 100 μM per 25 μL amplification reaction mix. When the HLA Class I oligonucleotides are provided at a concentration of about 100 μM in an amplification reaction mix of 25 μL, the DNA sample may be provided at an amount of 60 ng or more. It is apparent that these numbers can be scaled relative to each other.

In step b), the HLA Class II oligonucleotides may be provided at a concentration of about 5-100 μM, suitably about 10-50 μM, most suitably about 20 μM per 25 μL amplification reaction. When the HLA Class II oligonucleotides are provided at a concentration of about 20 μM in an amplification reaction mix of 25 μL, the DNA sample is provided at an amount of 20 ng or more, such as 60 ng or more. It is apparent that these numbers can be scaled relative to each other.

In step a), the oligonucleotides may comprise oligonucleotides of SEQ ID NOs: 1-11, 16-35 and 37-42 or variants thereof. The set of oligonucleotides may further comprise one or more of oligonucleotides of SEQ ID NOs: 12, 13, 14, 15 and 36 or variants thereof. The set of oligonucleotides may comprise oligonucleotides of SEQ ID NOs: 1-42.

If HLA class I is being typed preferably the oligonucleotides used comprise at least oligonucleotides of SEQ ID Nos: 1-6 or variants thereof. If HLA class II is being typed preferably the oligonucleotides used comprise at least oligonucleotides of SEQ ID Nos: 7-11, 16-35 and 37-42 or variants thereof, one or more of oligonucleotides of SEQ ID NOs: 12, 13, 14, 15 and 36 or variants thereof may also be used.

The DNA sample may be a sample of DNA from a human subject. The DNA of the sample may have been extracted from a blood or tissue sample obtained from the subject.

In step b) of the method, the amplification method may comprise the use of a thermocycling profile. In particular, cycling conditions may be as follows:
i) about 95° C. for about 2 minutes;
ii) about 30 cycles, such as between 20 and 40 cycles, of: about 94° C. for about 30 seconds and about 65° C. for between about 4 and about 10 minutes, such as 4 minutes, 5 minutes, 6 minutes, 7 minutes, 8 minutes, 9 minutes, 10 minutes; and
iii) a final extension at about 72° C. for about 10 minutes.

In step b) of the method, the amplification method may comprise or consist of the use of a thermocycling profile. In particular, cycling conditions may be as follows:
i) 95° C. for 2 minutes;
ii) 30 cycles of: 94° C. for 30 seconds and 65° C. for between 4 and 10 minutes, such as 4 minutes, 5 minutes, 6 minutes, 7 minutes, 8 minutes, 9 minutes, 10 minutes; and
iii) a final extension step at 72° C. for 10 minutes.

In step b) of the method, the amplification method may consist of the use of a thermocycling profile. In particular, cycling conditions may be as follows:
i) 95° C. for 2 minutes;
ii) 30 cycles of: 94° C. for 30 seconds and 65° C. for 10 minutes; and
iii) a final extension step at 72° C. for 10 minutes.

Preferably all DNA amplification reactions are performed in the same thermocycler. However, each amplification reaction can also be performed independently.

The extension temperature depends on the DNA polymerase used. Usually, this temperature is about 65-72° C. However, some DNA polymerases may require adjustments. The extension time depends on the length of the amplicon and the speed of the polymerase and can be easily determined by the skilled person.

The method may also comprise one or more of the steps of: end repairing of the amplicons, adding a molecular barcode ‘tail’ to the amplicon, ‘clean-up’ of the amplicons, sorting the amplicons by size, and amplicon quantification.

In step c) of the method, the sequences of amplicons may be determined using a next generation sequencing (NGS) method, for example Oxford Nanopore® Technology or Illumina Technology®. All NGS methods are well known by the skilled person and can be easily performed according to the manufacturer's instructions.

The method may further comprise comparing the determined sequences of the amplicons with the DNA sequences of known HLA types, possibly using bioinformatics. The sequences can be analyzed using suitable software, such as software that is able to filter out related sequence reads (such as other unwanted HLA genes) that could be co-amplified with the target sequences. The software can be used to merge sequences together, to compare to HLA sequences database and to propose a genotype for each loci. Once the DNA sequences have been obtained, the assignment of genotypes at each locus is performed by comparing said sequences with the DNA sequences of known reference HLA types. Null alleles as well as new alleles can also be detected.

The method may also comprise haplotype phasing, and/or identification of homozygosity. For example, the derivation of haplotypes may be achieved via phasing of maternal and paternal contributions to alleles using computational techniques. Similar techniques may be used for identifying runs of homozygosity, which are one parent's contribution to the allele, or where the biological mother and father have the same allele at a given point.

The HLA typing referred to in any aspects of the invention may be to identify a suitable donor and/or recipient of a transplant, for paternity testing, for identifying the HLA type for determination of epitope binding capability in neo-antigen prediction, or for diagnosing an immune disorder such as ankylosing spondylitis.

The transplant may be a kidney transplant, heart transplant, bone marrow transplant, stem cell transplant, liver transplant, lung transplant, pancreas transplant, small bowel transplant, or uterine transplant.

Thus, the method may further comprise step d), in which a suitable transplant donor and/or recipient is identified, if at least the first fields match between donor and recipient, and as many subsequent fields as possible. This is because the risk of rejection decreases as the numbers of mismatches decreases (http://www.ctstransplant.org).

The invention solves the problem of phase ambiguity and detection of all polymorphisms such as single-nucleotide polymorphisms (SNPs) or indels that could result in null alleles, via amplification and sequencing the entire HLA loci, such that artificial phasing is unnecessary.

The technology described provides the ability to quickly and relatively cheaply perform HLA typing to an extremely high resolution, in order to identify HLA matched donors and recipients in transplant situations, reducing costs and transplant wastage (such as donated organs) due to the length of time current HLA typing takes in the clinic. Another advantage of the technology described herein is that inherent phasing ambiguities present in Sanger sequencing can be eliminated, the reads can be separated and assembled into phased consensuses, i.e from each allele. This allows the resolution of the entire HLA region to four-field resolution, picking up all sequence novelties and SNPs, whilst being able to phase the reads completely, so that each allele is correctly separated. Thus, an accurate HLA match can be identified quickly and confidently. In addition, the correct phasing allows the determination of lineage for matches; ie identifying one parent's lineage or the other as having the higher chance of success of being a HLA match for a transplant.

Currently, finding the best HLA match for a transplant generally means that the nucleotide sequences of both recipients and provisional donors are determined either by Sanger capillary or by NGS. Sanger sequencing can produce 1000 base-pairs long reads, but the signals from the two chromosomes are mixed. Therefore, there is an inherent phase ambiguity despite the long resulting reads. On the other hand, while reads from next-generation sequencers are from different chromosomes, their length are usually behind the stretch of Sanger traces, expected to be in the range of 4-500 base pairs that on average is 454, and 2×150 or 2×250 base pairs for Illumina sequencers. This again increases ambiguity: if the allele pair to be typed has a homozygous sequence region that is longer than the average read length and the insert between the pairs (the distance between the end of the read generated by the forward primer and the end of the read generated by the reverse primer), the phase cannot be resolved. Instead of an allele pair, only a list of possible alleles is obtained having similar nucleotide sequences but possibly different expressed proteins. Using the best sampling, targeting, and amplification technology combined with the latest HLA typing bioinformatics workflow can lead to ambiguity, when the two alleles of a heterozygous sample cannot be separated. Other sources of ambiguity from existing methods include lost homozygous stretches, PCR dropouts and imbalance, PCR crossover, and missing coverage (37).

The development of long read technology, as described herein, allows a solution to these problems. Long read sequencing of the HLA region has considerable advantages as the haploblock structure is maintained as with other genomic regions allowing accurate resolution of HLA alleles using haplotype inference (14) and techniques such as population reference graphing (15).

Development of an assay that provides “whole gene” sequencing of the HLA region, along with high resolution reconstruction of the alleles (known and novel) within it, phasing into maternal and paternal haplotypes and identification of regions of homozygosity, all within a cost effective, rapid and portable test has the potential to change the field of HLA diagnostics making this type of testing available to all.

One such use of the technology described herein could be its use with existing nanopore technology. A unique technical feature of nanopore sequencing is its scalability: from rapid, one sample, single gene sequencing through a single flow cell to high volume, whole genome sequencing. The method is remarkably cost-effective even for a single sample which means not having to resort to sequencing in large batches. Thus, for full gene HLA sequencing this could mean a fast turn-around for individual patients or recipient/donor pairs, including in a near-patient setting, to multiplex testing of large cohorts, and anything in between. The single molecule sequencing reads full length genes in real time so includes any DNA variations (in phase) that, for instance, correspond to expression level or other phenotypes (16).

In the field this could translate to simple and effective HLA typing requiring only relatively small pieces of equipment, of particular importance in remote areas, needing only the movement of data rather than movement of DNA or blood samples. For example, this approach could utilise the portability of nanopore sequencing, coupled to a laptop computer and portable PCR equipment to allow HLA typing in resource poor conditions. Results and typing could be achieved much quicker than currently possible, and the wastage of organs and tissues, from long testing which affects the quality of a given organ or tissue, to undertaking transplants which are rejected, would be greatly reduced. The cost of HLA typing would also be reduced significantly, sometimes by more than 90-95% compared to conventional HLA typing.

### Definitions

The term “allele” as used herein, refers to one of the alternative forms of a genetic locus. As used herein, the term “locus” refers to the position on a chromosome of a particular gene or allele.

The term “genotype” as used herein, refers to a description of the alleles of a gene or a plurality of genes contained in an individual or in a sample from said individual.

The expression “determining the HLA genotype” as used herein refers to determining the HLA polymorphisms present in the individual alleles of a subject.

The term “DNA sample” refers to a sample containing human genomic DNA obtained from a subject.

The term “primer” or “amplification primers” as used herein refers to an oligonucleotide that is capable of selectively hybridizing to a target nucleic acid or “template”, more particularly capable of annealing to a DNA region adjacent to a target sequence to be amplified, and provides a point of initiation for template-directed synthesis of a polynucleotide complementary to the template catalysed by a polymerase enzyme such as a DNA polymerase (polymerase chain reaction amplification). The primer is preferably a single-stranded oligo-deoxyribonucleotide. An amplification primer is typically 15 to 40 nucleotides in length, preferably 15 to 30 nucleotides in length. The amplification primer may comprise a region being complementary to the HLA sequence of interest and a region that is not complementary to the HLA sequence of interest. In this case, the region complementary to the HLA sequence of interest is at least 15 nucleotides in length. Primers are often obtained as synthesized molecules and can be designed with wide range of molecular modifications, in particular at their 5′- or 3′-terminus.

As used herein, the term “truncated” as it relates to an oligonucleotide, refers to an oligonucleotide wherein, by comparison to the reference sequence, e.g. one of the sequences set forth in SEQ ID NOs: 1-42, one or several nucleotides are missing at the 5′ and/or 3′ terminus.

The term “DNA amplification”, as used herein, refers to an enzymatic process of extension of nucleic acid molecules that needs polymerase enzyme, template molecule annealed with amplification primers as well as nucleotides and adequate environmental conditions. Examples of amplification techniques include, but are not limited to, polymerase chain reaction (PCR), modified PCR techniques and ligase chain reaction (LCR). Typically, the segment is defined by a forward primer and a reverse primer that hybridize to the 5′ end and 3′ end of the segment to be amplified. Conditions and reagents for primer extension reactions are well known in the art (see for example Sambrook et al. Molecular Cloning, A Laboratory Manual, Third Edition, Cold Spring Harbor Laboratory Press, 2000, and Ausubel et al., Current Protocols in Molecular Biology, John Wiley & Sons, N Y, 1998). Amplification reaction can comprise thermal-cycling or can be performed isothermally. Preferably the primer-dependent DNA amplification reaction is a polymerase chain reaction (PCR). Preferably, PCR is performed in a thermocycler.

The term “polymerase chain reaction” or “PCR” as used herein refers to a method for amplifying a DNA sequence using a heat-stable DNA polymerase and a set of amplification primers in a cyclical reaction where the annealing of primers, synthesis of progeny strand DNA and denaturation of the duplexes, are each conducted at different temperatures. Because the newly synthesized DNA strands can subsequently serve as additional templates for the same primer sequences, successive rounds of primer annealing, strand elongation and dissociation produce rapid amplification of the target sequence.

As used herein, the term “amplification reaction mixture” refers to a mixture comprising all reagents needed for performing primer-dependent DNA amplification reaction. Typically, this mixture comprises a DNA polymerase, a set of amplification primers, an appropriate buffer and dNTPs.

As used herein, the term “DNA polymerase” refers to an enzyme that is essential for elongation of amplification primers in nucleic acid templates. The skilled person may easily choose a convenient polymerase enzyme based on its characteristics such as efficiency, processivity or fidelity. Preferably, the polymerase is a high-fidelity and heat-stable polymerase.

The term “amplicon” or “amplification product” as used herein refers to a fragment of DNA spanned within a pair of amplification primers, this fragment being amplified exponentially by a DNA polymerase. An amplicon can be single-stranded or double-stranded.

The expression “determining the sequence” as used herein, refers to the process of determining the identity of nucleotide bases at each position along the length of a polynucleotide. Any sequencing method can be used in the present invention.

As used in this specification, the term “about” may refer to a range of values±10% of the specified value. For example, “about 20” may include ±10% of 20, and refer to from 18 to 22. Preferably, the term “about” may refer to a range of values±5% of the specified value.

As used herein, the term “Sequence identity” or “similarity” refers to the identity between two or more nucleic acid sequences or between two or more amino acid sequences. This can be measured in terms of percentage identity; the higher the percentage, the more identical the sequences are. Homologs or orthologs of nucleic acid or amino acid sequences possess a relatively high degree of sequence identity/similarity when aligned using standard methods. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations. The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, MD 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Preferably, the methods of the invention are in vitro or ex vivo methods.

HLA-A, HLA-B and HLA-C are the three major types of human MHC class I cell surface antigen-presenting proteins. They play a central role in the immune system by presenting peptides derived from the endoplasmic reticulum lumen and are expressed in nearly all cells. These receptors are heterodimers and are composed of a heavy a chain and a light chain (an invariant β2 microglobulin molecule coded for by a separate region of the human genome). The HLA-A gene (Gene ID: 3105) contain 8 coding exons, the HLA-B gene (Gene ID: 3106) and the HLA-C gene (Gene ID: 3107) contain 7 coding exons.

HLA class II molecules are heterodimers consisting of an alpha chain and a beta chain, both anchored in the membrane. They play a central role in the immune system by presenting peptides derived from extracellular proteins. Class II molecules are expressed in antigen presenting cells (e.g. B lymphocytes, dendritic cells, macrophages).

HLA-DRB1 (Gene ID: 3123), HLA-DRB3 (Gene ID: 3125), HLA-DRB4 (Gene ID: 3126) and HLA-DRB5 (Gene ID: 3127) belong to the HLA class II beta chain paralogs. The heterodimers consist of an alpha chain (DRA) and a beta chain (DRB). The beta chain is approximately 26-28 kDa and is encoded by 6 exons.

HLA-DQA1 (Gene ID: 3117) belongs to the HLA class II alpha chain paralogues. The heterodimers consist of an alpha chain (DQA) and a beta chain (DQB). The alpha chain is approximately 33-35 kDa and is encoded by 4 coding exons.

HLA-DQB 1 (Gene ID: 3119) belongs to the HLA class II beta chain paralogs. The beta chain is approximately 26-28 kDa and is encoded by 5 coding exons.

HLA-DPB1 (Gene ID: 3115) belongs to the HLA class II beta chain paralogues. The heterodimers consist of an alpha chain (DP A) and a beta chain (DPB). The beta chain is approximately 26-28 kDa and is encoded by 5 coding exons.

The skilled man will appreciate that preferred features of any one embodiment and/or aspect of the invention may be applied to all other embodiments and/or aspects of the invention.

The present invention will be further described in more detail, by way of example only, with reference to the following figures:

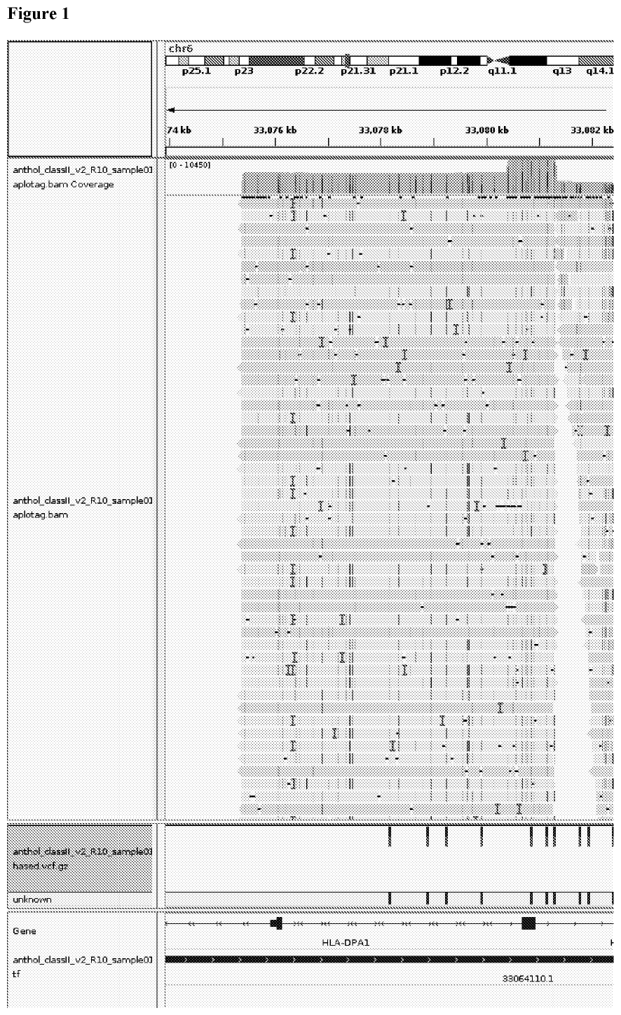

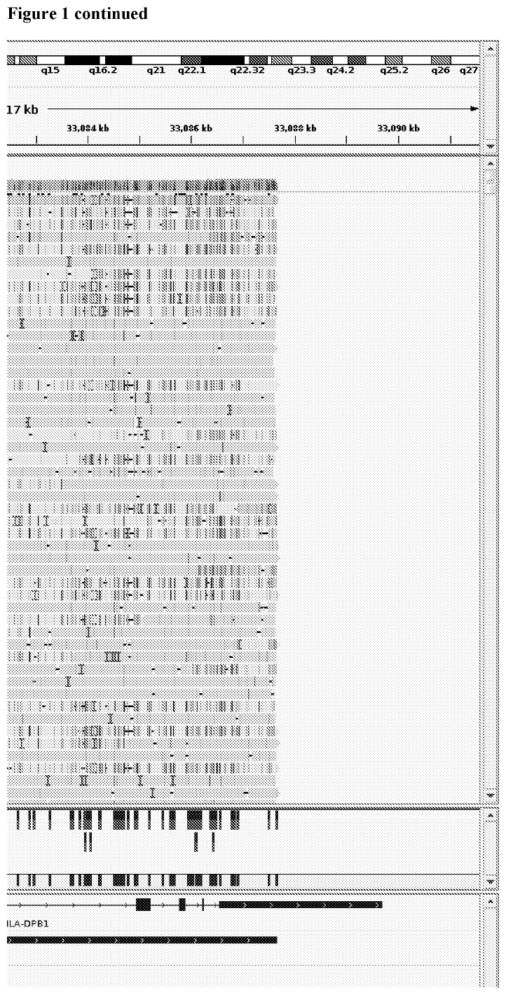

FIG. 1—is a plot from the software programme Integrated Genome Viewer (IGV) showing the region of the HLA-DPB1 gene. Within this the blue bars represent reads aligned to the HLA-DPB1 gene that are contributed by one parent and the green bars represent reads contributed from the other parent

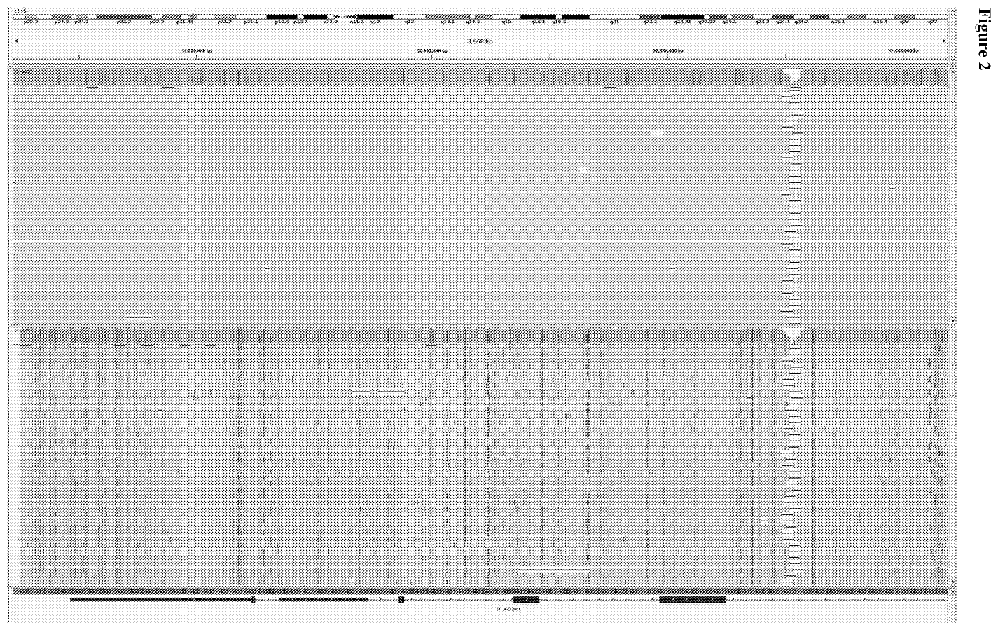

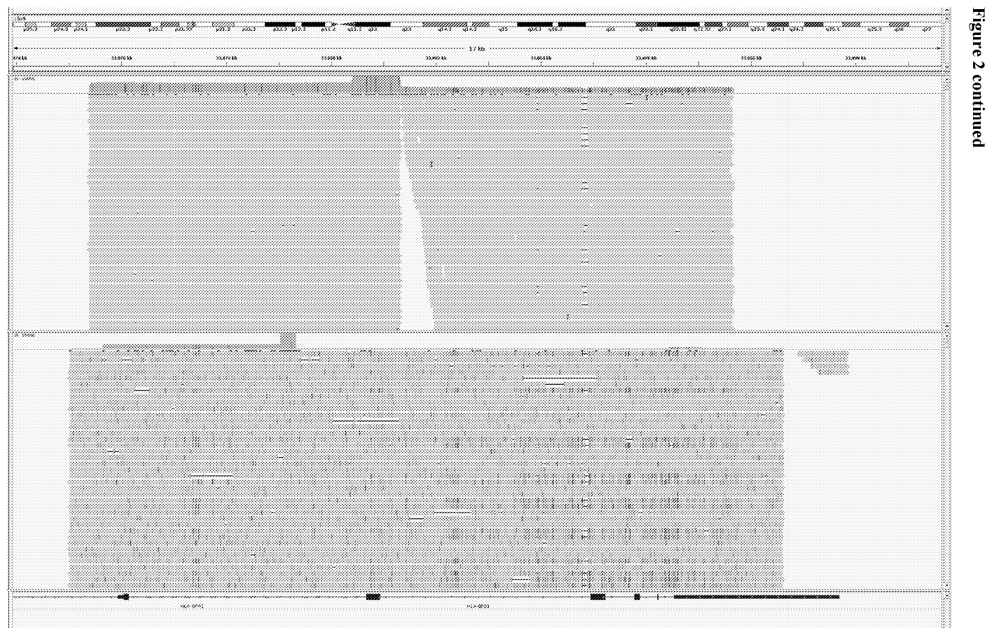

FIG. 2—is an IGV plot showing difference in single base mismatches and insertions/deletions (highlighted by coloured lines) in Top panel=HLA-DRB1; Middle panel=HLA-DPB1; Bottom panel=HLA-DRB5

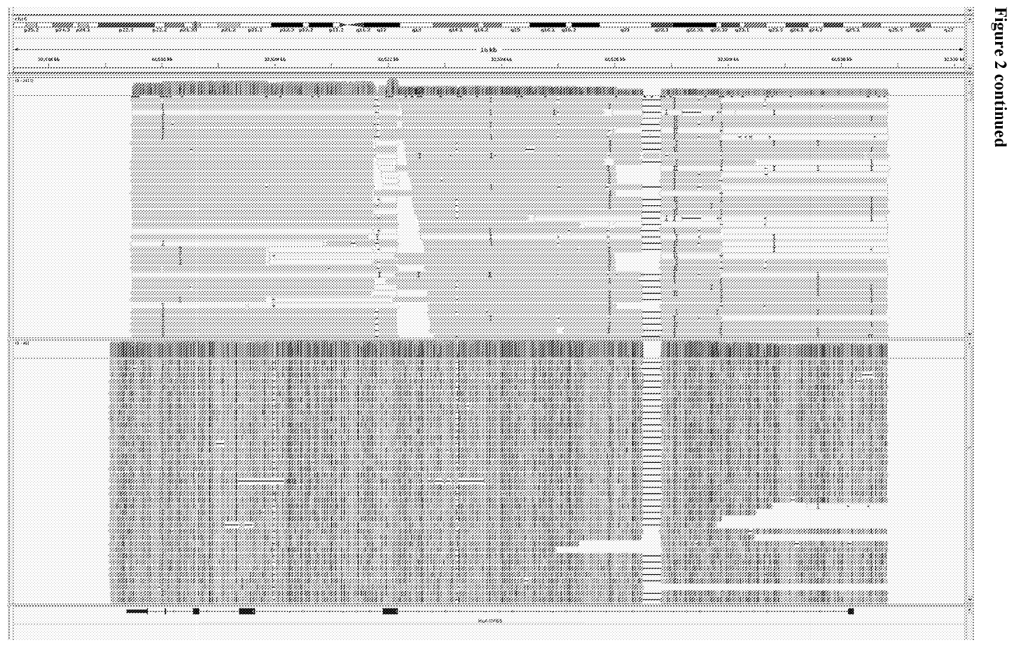

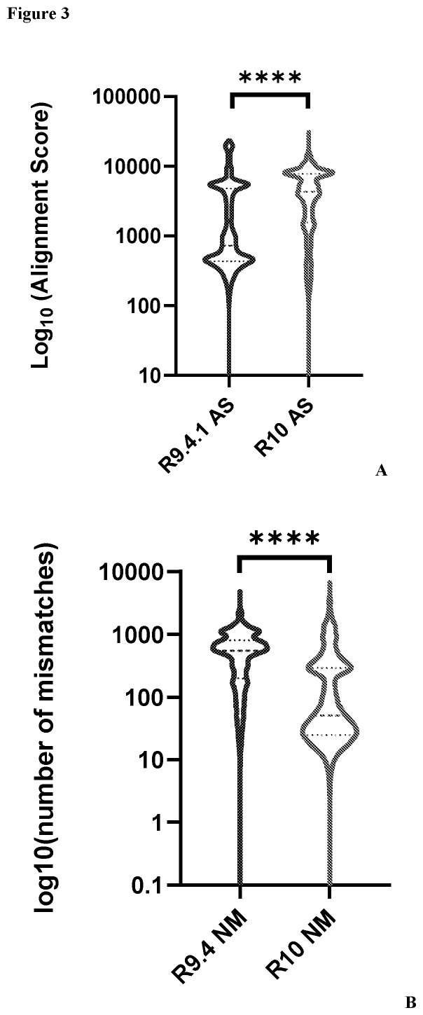

FIG. 3—shows violin and whisker plots of log 10 of: Left—the alignment score (higher is better) for a representative sample comparing R9.4.1 pore (blue—left of the plot) and R10 pore (red—right of the plot). Right—the number of mismatches (lower is better) for a representative sample comparing R9.4.1 pore (blue) and R10 pore (red).

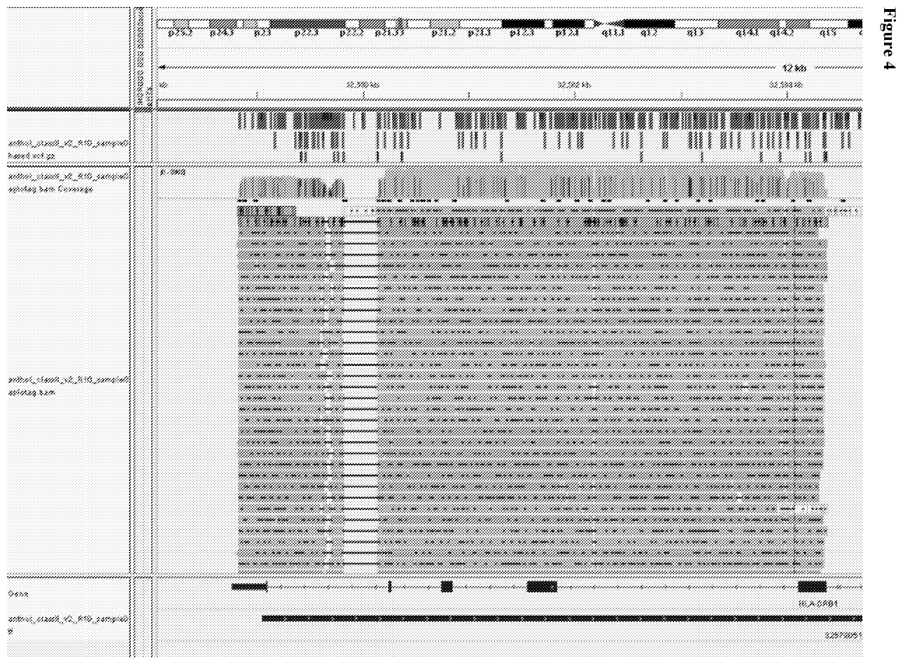

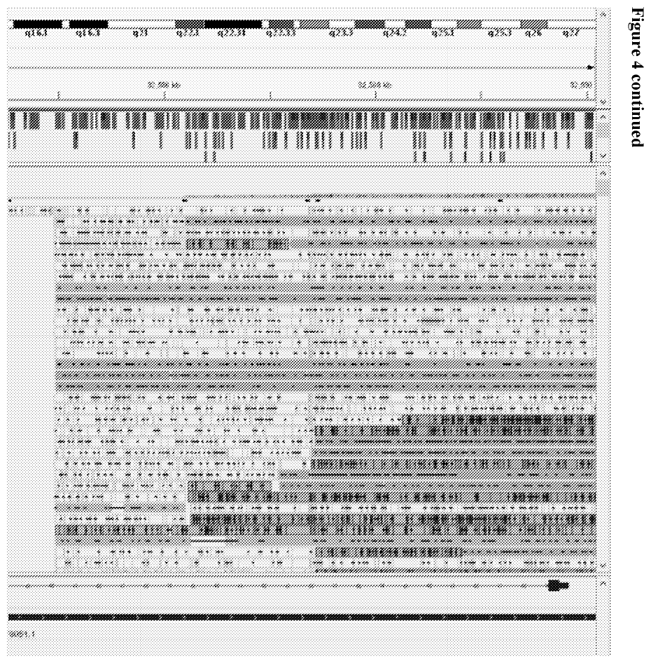

FIG. 4—shows am IGV plot showing that HLA-DRB1 is homozygous, represented by the VCF allele call plot (panel below ideogram) is composed of mostly homozygous (red) SNPs and occasional heterozygous (blue) SNPs.

### METHODS

Patient Samples Anonymised patient samples from organ donors were received from NHS Blood and Transplant under ethical approval (05/Q1605/66). Samples consisted of whole blood taken for routine HLA typing.

A further set of samples (the Frederick Hutchinson HLA Anthropology Panel) was also chosen that represents 15 samples from different regions of the world allowing us to understand the applicability of the assay to non CEPH samples and resolve unusual alleles.

### DNA Extraction

DNA extraction was performed using the Qiagen DNEasy kit using the standard manufacturers protocol. DNA was quantified on the Qubit broad range v3 DNA assay (for quantity) and Agilent Tapestation & Nanodrop (for DNA quality). DNA from the Frederick Hutchinson Centre was supplied pre-extracted but was quantified prior to use using the same methodology.

### HLA Reference Typing

Donor DNA was typed initially PCR-SSP (LinkSēq™, supplied by One Lambda) and/or SSO (Lifecodes, supplied by Imucor) as part of standard patient care. For Illumina based NGS typing, pre-amplification Fluorometic DNA quantitation was performed using the Qubit Broad Range kits (Thermo Fisher, UK). Prior to amplification genomic DNA was diluted to a concentration of 25 ng/μL. HLA loci were amplified using the AllType™ (One Lambda, USA) 11 locus kit, amplifiying HLA-A, -B, -C, DRB1, -DRB345, -DQA1, -DQB1, -DPA1 and -DPB1 in a multiplex PCR. Post amplification, products were purified using AMPure XP® (Agencourt, USA) beads and fluorometric quantitation was repeated using the Qubit (Invitrogen) High Sensitivity kit (dsDNA HS assay).

Amplicons were normalised, then enzymatically fragmented. Barcode ligation was followed by size selection (AMPure XP® beads), resulting in products of optimal size (300-1000 bp). A secondary amplification was performed prior to subsequent purification (AMPure XP® beads), quantification (Qubit dsDNA HS assay) and final equimolar pooling. The pooled library was denatured with NaOH (20%) and loaded onto an Illumina Micro Flowcell onto the MiSeq platform (Illumina, USA). HLA types were analysed using the Type Stream Visual version 1.2 (One Lambda, USA) software.

### HLA—Class I

Primer sequences are shown in Table 1 (SEQ ID NOs: 1-6). Amplicons for Class I HLA targets (whole gene including exon, intron and UTRs of HLA A, B, C, E,F and G) were generated in a multiplex reaction using the following conditions: 25 μL PCR reactions were performed using 60 ng DNA, 100 μM primer mix, 1× GoTaq Long (Promega, UK). HLA-E to G were not used in downstream analysis as no reference data existed for these genes. The cycling conditions were as follows: 95 C for 2 min followed by 30 cycles of 94 C for 30 sec and 65 for 4 min, with a final extension of 10 mins at 72 C.

### HLA Class II

Primer sequences are shown in Table 2 (SEQ ID NOs: 7-42). Amplicons for Class II (whole gene including exon, intron and UTRs of DRB1, DQB1, DQA1, DPA1 and DPB1) were generated with primers mixes as shown in table 2 using the following conditions: 25 μL PCR reactions were performed using 60 ng DNA, 20 μM primer mix, lx GoTaq Long (Promega, UK). The cycling conditions were as follows: 95 C for 2 min followed by 30 cycles of 94 C for 30 sec and 65 C for 5/7/9/10 min, with a final extension of 10 min at 72 C. Amplicons were then quantified by Qubit (Thermo Fisher Scientific, UK) according to the manufactures instructions and pooled in equimolar amounts for sequencing.

Custom primer design was also carried out for risk alleles in APOL1 that predispose to focal segmental glomerulosclerosis in African patients. The risk alleles were rs73885319 (GRCh38 Chr22:36265860) and rs60910145 (GRCh38 Chr22: 36265988). The PCR primers for this region were spiked into the HLA region as proof of concept.

### Library Preparation & Sequencing

Barcoded libraries were generated using the native barcoding (EXP-NBD104, EXP-NBD114) and sequencing by ligations kits (SQK-LSK109) from Oxford Nanopore. Briefly 1.3 μg of amplicon pools were end repaired and a tailed using NEBNext Ultra II module E7546 (3.5 μL End Repair Buffer, 2 ul FFPE repair mix, 3.5 μL Ultra II end-prep reaction buffer and 3 μL of Ultra II end-prep enzyme mix to 1.3 μg DNA in a total of reaction volume of 60 ul). This was incubated at 20 C for 5 min followed by 65 C for 5 min. Clean up was performed using AMPure XP beads (Beckman Coulter) in a 1× ratio. Quantification was performed using fluorimetry (Qubit) and 500 ng taken through to barcode ligation.

Native barcodes were ligated to 500 ng end-repaired/tailed DNA using NEB blunt/TA ligase M0367 (2.5 μL Native barcode, 25 μL Blunt/TA Ligase Master mIx to 500 ng DNA in a total volume of 50 ul). Following a 10 min incubation at room temperature the barcode ligated DNA was cleaned using AMPure XP beads (Beckman Coulter) in a 1× ratio. DNA quantification was performed using fluorimetry (Qubit) and a pool of all samples created with an overall concentration of 700 ng. To reduce the volume a further clean up was performed using 2.5× AMPure beads and eluting into 65 μL.

Adaptors were ligated by adding 20 μL barcode adaptor mix (Oxford Nanopore) 20 μL quick ligation buffer and 10 ul T4 ligase (NEB Module E6056). Following a 10 minute incubation at room temperature the adaptor ligated DNA was cleaned using AMPure beads in a 0.4× ratio and washed using Long Fragment Buffer (Oxford Nanopore) before eluting in 15 μL of elution buffer (Oxford Nanopore). Final quantification by fluorometry (Qubit) was performed and 30 fmol DNA prepared for sequencing according to the manufacturers instructions (Oxford Nanopore).

Sequencing was performed on a MinION R9.4.1 flow cell (MIN-106), a MinION R10 flow cell and a MinIon R9.4.1 Flongle flow cell and run for 8 hours using live basecalling, files were outputted in Fast5 and Fastq format.

### Bioinformatics Analysis

All data analysis was carried out on an Ubuntu 18.04LTS server (with 16 cores and 256 GB memory) and the University of Birmingham BEAR High Performance Computing (Bear-HPC) facility. The jobs submitted to the BEAR HPC facility utilised 32 cores and 256 GB of system memory with a wall time of 30 minutes per sample. Raw data underwent run management with MinKnow v19.05.0 and basecalling using the Guppy 3.1.5+781ed57 basecaller using standard parameters. Quality control plots were generated with NanoPlot 1.26.3 (23). Basecalled FASTQ files were demultiplexed using Guppy barcoder 3.1.5+781ed57 (parameters: -t 32 --trim_barcodes --require_barcodes_both_ends -q 0 --compress_fastq).

Binned reads were aligned to the Illumina Platinum GRCh38 reference genome using MiniMap v2.12 (parameter: -ax map-ont, setting a default mismatch penalty of 4) (24), sorted and indexed using Samtools 1.3.1 using htslib 1.31. (25, 26). The aligned BAM file was then input into the HLA-LA*v1.2 pipeline (27). Output at 4 field resolution (via the R1_bestguess.txt output) was taken as consensus output to compare to reference Illumina/Sanger/SSP calls. For FSGS risk alleles, the aligned BAM files were filtered for the region of interest (GRCh38 Chr22: 36265800-36266100) and then variant calling was performed using FreeBayes v1.0.0 (28) outputting all sites in gVCF mode.

Haplotype phasing of the HLA amplicon data was carried out using WhatsHap v.0.18 (29). Initially variant calls for the amplicon data was produced using Freebayes (parameters: -C 2-0 -O -q 20 -z 0.10 -E 0 -X -u -p 2 -F 0.6), then using WhatsHap to produce a phased variant call file (parameters: -o phases.vcf input.bam). A phased haplotype GTF and a haplotagged BAM file were then produced (using the whatshap stats and whatshap haplotag commands respectively) for visualisation. For identification of homozygosity, visual inspection of the variant calls in IGV was carried out.

Concordance between reference and Nanopore sequenced HLA alleles was defined at each field level as to whether there was an exact match. If there was, this was marked as correct. The numbers of correct alleles were divided by the total number of reference fields present across all the samples (Supplementary data) If there was no 3rd or 4th field, the total number of fields was reduced by number of samples missing the 3rd/4th field.

### EXAMPLES

### Example 1—Rapid, Highly Accurate and Cost-Effective HLA Typing HLA Class I and Class II Alleles

### Data Delivery

For the NHSBT sample typing, in total 2.7GBases of sequencing data was produced, with a median read length of 3,377 bases, a read length N50 of 3,606 bases and a median read quality of 9.4. For the Anthropology panel sample typing a total of 3.8GBases of sequencing data was produced, with a median read length of 3,170 bases a read length N50 of 3,513 bases and a median read quality of 9.9. Run time was standardised at 8 hours for both panels. For the single Flongle sequenced sample, 43,266 reads with a median read length of 1,080 bases were produced with a total output of 110 megabases of sequence.

### Workflow

The multiplex long range PCR reaction took 150 minutes, followed by a modified LSK-109 protocol taking 30 minutes, followed by 120 min on the Nanopore system and 30 minutes of assembly of the HLA calls. The yield of the flowcells over the project determined the run time. Typically a run of 2 hours for a single sample on the Flongle (40 mb yield) and 50 minutes for 12 multiplexed samples on the MinIon (396 mb yield) allowed sufficient data for 500× coverage. The run time was therefore set at 2 hours.

### Class I & Class II HLA Call Accuracy

In preliminary analysis it was found that at least 500× coverage of each amplicon was required for accurate HLA calling, therefore in samples with low coverage these were rerun. For the 1st set of NHSBT samples, 11 samples underwent analysis for Class I alleles (Table 1). All samples were correct for first field, NHSBT Sample 1 had a reference BTS HLA-C allele of 7, for the MiSeq call it was C*07:02:01:03 (although the C*07:123 was given as the second option in the BTS typing) and for the Nanopore it was C*07:123.

For the second set of NHSBT samples, a more challenging set of two samples were chosen. Concordance for Class I and Class II calls was 100% with 0% error.

For the Anthropology panel, 15 samples underwent analysis for Class I and Class II alleles (Table 4). All samples were an exact match apart from sample IHW09376. For the single 2nd field error the reference call was HLA-B*27:05:02 and the Nanopore call HLA-B*27:110. This representations a single nucleotide change (G>A) and could represent a sequencing error for either method. For the class II alleles all samples were a match except IHW09021, where the reference for HLA-DRB1 was DRB1*03:02:01 and the MinIon call was 03:03. Examination of the raw data revealed that this was a sequence alignment error caused by an indel from Nanopore sequencing. When manual correction was applied the allele resolved correctly.

### FSGS/APOL1 Allele Calling

In order to understand the utility of the Nanopore system for SNP variants that may predispose to clinically relevant diseases, the G1 and G2 risk alleles for focal segmental glomerulosclerosis were spiked into the mix. The G1 alleles (rs73885319, Chr22: 36265860, NC_000022.10: g.36661906A>G and rs60910145, Chr22:36265988, NC_000022.10: g.36662034T>G) were called in all the NHSBT samples. Of the twelve samples, all had the A reference allele. The G2 allele is a 6 bp (rs71785313, Chr22: 36266000, NC_000022.10: g. 36662046_36662051 delTTATAA) deletion in APOL1. Of the twelve samples, the indel was not seen. Of note, several small common SNPs within 200 bp of the region of the SNPs of the APOL1 gene were observed, for example rs1403581130.

R9.4.1 vs. R10 Pores

As part of an early access programme, the project was given to the new R10 Nanopore to run HLA typing samples on (FIG. 1). The R10 was called using the identical pipeline to the R9 data and displayed significantly higher single base accuracy. In FIG. 2, all three panels demonstrate IGV plots of the R10 data (top of each panel) vs. R9 data (bottom of each panel) demonstrating a greatly reduced level of single base mismatches across the three HLA genes shown— HLA-DQB1 (top), HLA-DPB1 (middle) and the highly polymorphic HLA-DRB5. Interestingly, raw average MAPQ scores were similar between R10 and R9 (49 vs. 44) and base mapping quality scores (16.2 vs. 15.5) equivalent to base error rates of 2.4% vs. 2.8%. Median alignment score (AS, where higher is a better score) as reported by MiniMap2 was 4350 for the R10 pore vs. 722 for the R9.4.1 pore (Mann-Whitney p<0.0001, FIG. 3). Median number of mismatches (NM, where fewer mismatches is better) as reported by MiniMap2 was 51 for the R10 pore vs. 551 for the R9.4.1 pore (Mann Whitney p<0.0001, FIG. 3).

### Single Sample Calling on the Flongle Device

In order to understand whether the output of a miniaturised Nanopore device—the Flongle Flowcell—a single sample (NHSBT sample 27) was run on a R9.4.1 Flongle. Data output was 0.9 Gb and 100% accuracy was seen at 4 field level for both class I and class II fields for this sample.

### HLA Phasing & Identification of Homozygosity in HLA-DRB1

Identification of maternal and paternal contributions to HLA alleles is vital to identify runs of homozygosity which may affect organ matching, as well as being difficult to detect using short read technologies. In order to demonstrate the ability of nanopore long read sequencing to phase HLA as well as identifying runs of homozygosity, a single sample (Anthropology panel sample 1, IHW09377) was chosen for analysis. After variant calling with FreeBayes, haplogroups were generated with WhatsHap. For this sample, two haplogroups were derived for each sample, presumably the maternal and paternal contribution to the inherited HLA of the proband. This could be clearly seen in IGV for HLA-DRB1 (FIG. 1) by generating a haplogroup tagged BAM files. In this figure, the separate contributions from maternal and paternal alleles can be seen in the differently coloured reads (green for haplogroup 1, blue for haplogroup 2). Each haploblock spanned the entire amplicon, reinforcing the co-dominant inheritance of the HLA system. Visual inspection of sample IHW09377 in the anthropology panel revealed that HLA-DRB1 was homozygous (FIG. 4)

### Speed & Cost Effectiveness

The Nanopore based assay showed considerable speed-based advantages over conventional typing. DNA extraction took 1 hour, library preparation 3 hours and sequencing 4-20 hours depending on volume of sequence data required. Bioinformatics analysis took 1 hour on a 16 core Intel Xeon server with 256 GB of system memory running Ubuntu LTS 18.04, meaning that in total the assay could be run within 8 hours which is a considerable time saving over NGS and SSP methods. In terms of cost effectiveness, the method of the invention costs around £38 GBP compared to a typical commercial HLA typing which costs in the range of £300-800 GBP.

### Summary

Full length HLA typing using long range PCR and sequencing on a nanopore sequencing system is shown to be highly accurate using the methodology of the invention. It is also cheaper than the nearest alternative and feasible for deployment into the field using a “laboratory in a suitcase” approach. This approach uses the portability of nanopore sequencing, coupled to a laptop computer and portable PCR equipment to allow HLA typing in resource poor conditions.

Current methodologies for typing of HLA rely on highly specific, but not broad assays such as single site polymorphism (SSP) assays (24) that can sequence individual alleles but not provide in depth reconstruction of the entire region of interest. This means that for rarer alleles although SSP provide accuracy this is at the cost of a single assay that can be utilised for all patients. Long amplicons, provided by long range PCR have been previously performed using short read sequencing (25), however the present strategy coupled with the long read capability of the Nanopore system provides a unique ability to accurately understand the HLA region.

The use of long range PCR (26) has advantages in that the entire gene can be encompassed in one PCR reaction, allowing reconstruction of haplotypes (27) and accurate resolution of complex parts of the HLA region. It also requires limited sample input (typically 50 ng of genomic DNA). The longest PCR amplicon (>10 kb) requires over 10 minutes per cycle which means that a typical long range PCR reaction for HLA typing takes just over 3 hours. This methodology however has the advantage that is can be performed in relatively resource poor environments enabling its use in lower and middle income countries (LMIC). Thus, this strategy could be used as an alternative to expensive and slow out of country HLA typing.

The algorithm used for reconstruction of the HLA region (HLA-LA) here has significant advantages as it uses a population reference graph of HLA alleles (21) to accurately reconstruct the HLA region to high accuracy. The use of a cloud based infrastructure where nanopore sequencing data is uploaded from the field and HLA types called in real time may make using such a strategy even easier in the field. This has the advantage of centralised control of the algorithm and quality assurance.

For Class I concordance (to 4 field accuracy where it was available, otherwise 3 field) was 100% for all 33 samples. Class II concordance (to 4 field accuracy where it was available, otherwise 3 field) was 100% at the first field level and 97.8% at the 2nd/3rd/4th field level in all 33 samples. Phasing of maternal and paternal alleles, as well as phasing based identification of runs of homozygosity was demonstrated successfully

In summary, this methodology allows for four field resolution of all Class I and Class II alleles and effective phasing of parental alleles. It is cost effective, rapid and has many practical advantages.

TABLE 1
HLA Class I primers

| | Sense | | Anti-sense | | Amplicon |
|---|---|---|---|---|---|
| Target | Primer | Sequence | Primer | Sequence | Size (bp) |
| A | HLA- | ATCCTGGATACT | HLA- | CATCAACCTCT | 3398 |
| | A_F7 | CACGACGCGGAC | A_R8 | CATGGCAAGAA | |
| | | (SEQ ID NO: 1) | | TTT | |
| | | | | (SEQ ID NO: 2) | |
| B | HLA- | AGGTGAATGGCT | HLA- | AGAGTTTAATT | 4296 |
| | B_F3 | CTGAAAATTTGT | B_R3 | GTAATGCTGTT | |
| | | CTC | | TTGACACA | |
| | | (SEQ ID NO: 3) | | (SEQ ID NO: 4) | |
| C | HLA-CF | CAGCACGAAGAT | HLA-CR | TGAGGAAAAGG | 4906 |
| | | CACTGG AA | | AGCAGAG GA | |
| | | (SEQ ID NO: 5) | | (SEQ ID NO: 6) | |

TABLE 2
HLA Class II primers

| | | | Anti- | | | Ext. |
|---|---|---|---|---|---|---|
| | Sense | | sense | | Amplicon | time |
| Target | Primer | Sequence | primer | Sequence | Size (bp) | (min) |
| HLA- | DRB1_ | CTGCTGCTCCTT | DRB1_ | CTTCTGGCTGTT | 6146- | 10 |
| DRB1 | PE2-F1 | GAGGCATCCAC | PE2-R1 | CCAGTACTCGG | 11,478 | |
| Mix 1 | | A | | CAT | | |
| | | (SEQ ID NO: 7) | | (SEQ ID NO: 8) | | |
| | DRB1_ | CTGCTACTCCTT | DRB1_ | CTTCTGGCTGTT | | |
| | PE2-F2 | GAGGCATCCAC | PE2-R2 | CCAGGACTCGG | | |
| | | A | | CGA | | |
| | | (SEQ ID NO: 9) | | (SEQ ID NO: 10) | | |
| | DRB1_ | CTGCTGCTCCC | DRB1_ | CTTCTGGCTGTT | | |
| | PE2-F3 | TGAGGCATCCA | PE2-R3 | CCAGTACTCAG | | |
| | | CA | | CGT | | |
| | | (SEQ ID NO: 11) | | (SEQ ID NO: 12) | | |
| | | | DRB1_ | CTTCTGGCTGTT | | |
| | | | PE2-R4 | CCAGTACTCCT | | |
| | | | | CAT | | |
| | | | | (SEQ ID NO: 13) | | |
| | | | DRB1_ | CTTCTGGCTGTT | | |
| | | | PE2-R5 | CCAGTGCTCCG | | |
| | | | | CAG | | |
| | | | | (SEQ ID NO: 14) | | |
| | | | DRB1_ | CTTCTGGCTGTT | | |
| | | | PE2-R6 | CCAGTACTCGG | | |
| | | | | CGC | | |
| | | | | (SEQ ID NO: 15) | | |
| HLA- | DRB1- | GCACGTTTCTT | DRB1- | ATGCACGGGAG | 5106 to | 10 |
| DRB1- | E2-1.1- | GTGGCAGCTTA | E2-12- | GCCATACGGT | 6218 | |
| mix2 | F | AGTT | R | (SEQ ID NO: 17) | | |
| | | (SEQ ID NO: 16) | | | | |
| | DRB1- | GCACGTTTCTT | DRB1- | ATGCACAGGAG | | |
| | E2-1.2- | GTGGCAGCTAA | E2- | GCCATAGGGT | | |
| | F | AGTT | 3568-R | (SEQ ID NO: 19) | | |
| | | (SEQ ID NO: 18) | | | | |
| | DRB1- | TTTCCTGTGGC | DRB1- | ATGCATGGGAG | | |
| | E2-2-F | AGCCTAAGAGG | E2-4-R | GCAGGAAGCA | | |
| | | (SEQ ID NO: 20) | | (SEQ ID NO: 21) | | |
| | DRB1- | CACAGCACGTT | DRB1- | CAGATGCATGG | | |
| | E2- | TCTTGGAGTAC | E2-7- | GAGGCAGGAAG | | |
| | 3568-F | TC | R2 | CG | | |
| | | (SEQ ID NO: 22) | | (SEQ ID NO: 23) | | |
| | DRB1- | AGCACGTTTCT | DRB1- | ATGCATGGGAG | | |
| | E2-4-F | TGGAGCAGGTT | E2-9-R | GCAGGAAGCG | | |
| | | AAACA | | (SEQ ID NO: 25) | | |
| | | (SEQ ID NO: 24) | | | | |
| | DRB1- | CACAGCACGTT | DRB1- | TGGAATGTCTA | | |
| | E2-7-F4 | TCCTGTGGCAG | E2-10- | AAGCAAGCTAT | | |
| | | GG | R | TTAACATATGT | | |
| | | (SEQ ID NO: 26) | | (SEQ ID NO: 27) | | |
| | DRB1- | CACAGCACGTT | | | | |
| | E2-9-F | TCTTGAAGCAG | | | | |
| | | GA | | | | |
| | | (SEQ ID NO: 28) | | | | |
| | DRB1- | ACAGCACGTTT | | | | |
| | E2-10-F | CTTGGAGGAGG | | | | |
| | | T | | | | |
| | | (SEQ ID NO: 29) | | | | |
| HLA- | HLA- | GCCAGGGAGGG | HLA- | ATCCAGTGGAG | 7488 | 7 |
| DQA1 | DQA1_ | AAATCAACT | DQA1_ | GACACAGCAC | | |
| | F2 | (SEQ ID NO: 30) | R2 | (SEQ ID NO: 31) | | |
| HLA- | DQB1- | AAGAAACAAAC | DQB1- | TAGTATTGCCC | 9093 | 9 |
| DQB1 | F3.1 | TGCCCCTTACA | | CTAGTCACTGT | | |
| | | CC | R3.1 | CAAG | | |
| | | (SEQ ID NO: 32) | | (SEQ ID NO: 33) | | |
| | DQB1- | AAGAAACAAAC | DQB1- | TAGTACTGCCC | | |
| | F3.2 | TGCCCCTTATA | | CTAGTCACTGC | | |
| | | CC | R3.2 | CAAG | | |
| | | (SEQ ID NO: 34) | | (SEQ ID NO: 35) | | |
| | | | DQB1- | TAGTACTGTCC | | |
| | | | R3.3 | CTAGTCACTGC | | |
| | | | | CAAG | | |
| | | | | (SEQ ID NO: 36) | | |
| HLA- | HLA- | CTCTCTTGACC | HLA- | TTGGCCTCTTG | 9,709 | 9 |
| DPA1 | DPA1_ | ACGCTGGTACC | DPA1_ | GCTATACCTCT | | |
| | F1 | TA | R1 | TTT | | |
| | | (SEQ ID NO: 37) | | (SEQ ID NO: 38) | | |
| HLA- | DPB1_ | CCTCCTGACCC | DPB1 | CCATCTGCCCC | 5940 | 5 |
| DPB1 | pro-F2 | TGATGACAGTC | pro-R2 | TCAAGCACCTC | | |
| | | CT | | AA | | |
| | | (SEQ ID NO: 39) | | (SEQ ID NO: 40) | | |
| HLA- | HLA- | CTCAGTGCTCG | HLA- | CTCAGTGCTCG | 7272 | 5 |
| DPB1 | DPB1_ | CCCCTCCCTAG | DPB1_ | CCCCTCCCTAG | | |
| | F2 | TGAT | R2 | TGAT | | |
| | | (SEQ ID NO: 41) | | (SEQ ID NO: 42) | | |

TABLE 3
List of results for samples within NHSBT experiment.

| IHW | | | | | | | |
|---|---|---|---|---|---|---|---|
| ID | Technique | A* | | B* | | C* | |
| 1 | Reference | A*24:02:01:01 | A*31:01:02:01 | B*07:02:01:01 | B*39:06:02:01 | C*07:02:01:01 | C*07:02:01:03 |
| | MinIon | A*24:02:01:01 | A*31:01:02:01 | B*07:02:01:01 | B*39:06:02:01 | C*07:02:01:01 | C*07:123 |
| | BTS | 24 | 31 | 7 | 39 | 7 | 7 |
| 2 | Reference | A*02:01:01:01 | A*11:01:01:01 | B*35:01:01:05 | B*40:01:02:01 | C*03:04:01:01 | C*04:01:01:05 |
| | MinIon | A*02:01:01:01 | A*11:01:01:01 | B*35:01:01:01 | B*40:01:02:01 | C*03:04:01:01 | C*04:01:01:05 |
| | BTS | 2 | 11 | 35 | 40:01 | 03:02 | 4 |
| 3 | Reference | A*01:01:01:01 | A*02:01:01:01 | B*08:01:01:01 | B*44:02:01:01 | C*05:01:01:02 | C*07:01:01:01 |
| | MinIon | A*01:01:01:01 | A*02:01:01:01 | B*08:01:01:01 | B*44:02:01:01 | C*05:01:01:02 | C*07:01:01:01 |
| | BTS | 1 | 2 | 8 | 44:02 | 5 | 7 |
| 5 | Reference | A*02:01:01:01 | A*03:01:01:01 | B*27:05:02:05 | B*44:02:01:01 | C*02:02:02:01 | C*05:01:01:02 |
| | MinIon | A*02:01:01:01 | A*03:01:01:01 | B*27:05:02:05 | B*44:02:01:01 | C*02:02:02:01 | C*05:01:01:02 |
| | BTS | 2 | 3 | 27 | 44:02 | 2 | 5 |
| 6 | Reference | A*02:01:01:01 | A*03:01:01:01 | B*40:01:02:01 | B*44:02:01:01 | C*03:04:01:01 | C*05:01:01:01 |
| | MinIon | A*02:01:01:01 | A*03:01:01:01 | B*40:01:02:01 | B*44:02:01:01 | C*03:04:01:01 | C*05:01:01:01 |
| | BTS | 2 | 3 | 40:01 | 44:02 | 03:02 | 5 |
| 7 | Reference | A*02:11:01:01 | A*11:01:01:01 | B*40:06:01:02 | B*51:01:01:12 | C*12:09 | C*15:02:01:01 |
| | MinIon | A*02:11:01:01 | A*11:01:01:01 | B*40:06:01:02 | B*51:01:01:12 | C*12:09 | C*15:02:01:01 |
| | BTS | 2 | 11 | 40:06 | 51 | 12 | 15 |
| 8 | Reference | A*02:01:01:01 | A*33:01:01:01 | B*44:03:01:02 | B*51:01:01:05 | C*02:02:02:01 | C*15:02:01:01 |
| | MinIon | A*02:01:01:01 | A*33:01:01:01 | B*44:03:01:02 | B*51:01:01:05 | C*02:02:02:01 | C*15:02:01:01 |
| | BTS | 2 | 33 | 44:03 | 51 | 02 | 15 |
| 9 | Reference | A*01:01:01:01 | A*68:01:02:02 | B*08:01:01:01 | B*35:08:01:01 | C*04:01:01:06 | C*07:141:02 |
| | MinIon | A*01:01:01:01 | A*68:01:02:02 | B*08:01:01:01 | B*35:08:01:01 | C*04:01:01:06 | C*07:141:02 |
| | BTS | 1 | 68 | 00 | 35 | 4 | 7 |
| 10 | Reference | A*01:01:01:01 | A*02:01:01:01 | B*08:01:01:01 | B*15:01:01:01 | C*03:04:01:01 | C*07:01:01:16 |
| | MinIon | A*01:01:01:01 | A*02:01:01:01 | B*08:01:01:01 | B*15:01:01:01 | C*03:04:01:01 | C*07:01:01:16 |
| | BTS | 1 | 2 | 8 | 15:01 | 03:02 | 7 |
| 11 | Reference | A*24:02:01:01 | A*25:01:01:01 | B*07:02:01:01 | B*18:01:01:01 | C*07:02:01:01 | C*12:03:01:01 |
| | MinIon | A*24:02:01:01 | A*25:01:01:01 | B*07:02:01:01 | B*18:01:01:01 | C*07:02:01:01 | C*12:03:01:01 |
| | BTS | 24 | 25 | 7 | 18 | 7 | 12 |
| 12 | Reference | A*11:01:01:01 | A*68:01:02:01 | B*07:02:01:01 | B*44:02:01:01 | C*05:01:01:02 | C*07:02:01:03 |
| | MinIon | A*11:01:01:01 | A*68:01:02:01 | B*07:02:01:01 | B*44:02:01:01 | C*05:01:01:02 | C*07:02:01:03 |
| | BTS | 11 | 68 | 7 | 44:02 | 5 | 7 |

RunID = internal run ID; Alternate ID = NHSBT Sample ID; Technique - reference: MinIon sequencing by NHSBT, MinIon = Nanopore based HLA typing, BTS = NHSBT serotyping derived allele. Font type represent accuracy of match - non-bold = all fields match; Bold = 2nd field mismatch; italic = 1st field mismatch

Field Concordance

| Field | Correct | Incorrect | Total | Percent |
|---|---|---|---|---|
| 1st | 66 | 0 | 66 | 100% |
| 2nd | 65 | 1 | 66 | 98% |
| 3rd | 64 | 1 | 65 | 98% |
| 4th | 64 | 1 | 65 | 98% |

Total Concordance

| Fields | Incorrect | | | | Ctotal | | | | Ttotal | Percentage |
|---|---|---|---|---|---|---|---|---|---|---|
| 1st | 66 | 88 | 66 | 27 | 247 | 88 | 66 | 27 | 247 | 100.0% |
| 2nd | 65 | 87 | 65 | 27 | 244 | 88 | 66 | 27 | 247 | 98.8% |
| 3rd | 64 | 81 | 60 | 26 | 231 | 82 | 61 | 26 | 234 | 98.7% |
| 4th | 64 | 63 | 23 | 23 | 173 | 64 | 24 | 23 | 176 | 98.3% |

TABLE 4
List of results for samples within Anthropology panel experiment.

| IHW ID | Technique | A* | | B* | |
|---|---|---|---|---|---|
| S20 | Reference | A*24:02:01:01 | A*31:01:02:01 | B*07:02:01:01 | |
| | MinIon | A*24:02:01:01 | A*31:01:02:01 | B*07:02:01:01 | |
| S27 | Reference | A*66:01:01:01 | A*74:01:01:01 | B*14:02:01:01 | B*15:03:01:01 |
| | MinIon | A*66:01:01:01 | A*74:01:01:01 | B*14:02:01:01 | B*15:03:01:01 |
| | Flongle | A*66:01:01:01 | A*74:01:01:01 | B*14:02:01:01 | B*15:03:01:01 |
| IHW09377 | Reference | A*02:01:01:01 | A*29:02:01:01 | B*27:09 | B*44:03:01:01 |
| | MinIon | A*02:01:01:01 | A*29:02:01:01 | B*27:09 | B*44:03:01:01 |
| IHW01093 | Reference | A*01:01:01:01 | A*68:01:02:02 | B*44:04 | B*15:01:01:01 |
| | MinIon | A*01:01:01:01 | A*68:01:02:02 | B*44:04 | B*15:01:01:01 |
| IHW09381 | Reference | A*02:06:01:01 | A*30:02:01:01 | B*39:08 | B*18:01:01:01 |
| | MinIon | A*02:06:01:01 | A*30:02:01:01 | B*39:08 | B*18:01:01:01 |
| IHW01141 | Reference | A*02:01:01:01 | A*01:01:01:01 | B*07:02:01 | B*08:01:01:01 |
| | MinIon | A*02:01:01:01 | A*01:01:01:01 | B*07:02:01 | B*08:01:01:01 |
| IHW09021 | Reference | A*30:01:01 | A*68:02:01:01 | B*42:01:01 | |
| | MinIon | A*30:01:01 | A*68:02:01:01 | B*42:01:01 | |
| IHW09107 | Reference | A*24:02:01:01 | A*24:02:43 | B*54:01:01 | |
| | MinIon | A*24:02:01:01 | A*24:02:43 | B*54:01:01 | |
| IHW09388 | Reference | A*11:01:01:01 | A*03:01:01:05 | B*40:01:02 | |
| | MinIon | A*11:01:01:01 | A*03:01:01:05 | B*40:01:02 | |
| IHW01175 | Reference | A*02:01:01:01 | A*02:05:01 | B*15:01:01:01 | B*49:01:01 |
| | MinIon | A*02:01:01:01 | A*02:05:01 | B*15:01:01:01 | B*49:01:01 |
| IHW09375 | Reference | A*33:01:01 | A*31:01:02:01 | B*14:02:01:01 | B*35:02:01 |
| | MinIon | A*33:01:01 | A*31:01:02:01 | B*14:02:01:01 | B*35:02:01 |
| IHW09376 | Reference | A*01:01:01:01 | | B*27:03 | B*27:05:02 |
| | MinIon | A*01:01:01:01 | | B*27:03 | B*27:110 |
| IHW09056 | Reference | A*02:01:01:01 | | B*35:03:01:01 | |
| | MinIon | A*02:01:01:01 | | B*35:03:01:01 | |
| IHW09367 | Reference | A*11:02:01 | A*02:03:01 | B*38:02:01 | B*46:01:01 |
| | MinIon | A*11:02:01 | A*02:03:01 | B*38:02:01 | B*46:01:01 |
| IHW09373 | Reference | A*68:02:01:01 | A*02:05:01 | B*58:01:01:01 | B*14:02:01:01 |
| | MinIon | A*68:02:01:01 | A*02:05:01 | B*58:01:01:01 | B*14:02:01:01 |
| IHW09024 | Reference | A*11:01:01:01 | A*02:06:01:01 | B*35:01:01:02 | B*15:01:01:01 |
| | MinIon | A*11:01:01:01 | A*02:06:01:01 | B*35:01:01:02 | B*15:01:01:01 |
| IHW09045 | Reference | A*03:01:01:01 | A*02:16 | B*51:01:01:01 | |
| | MinIon | A*03:01:01:01 | A*02:16 | B*51:01:01:01 | |

IHW ID = International Histocompatibility Workshop ID; Technique - reference: alleles supplied by IHW, MinIon = Nanopore based HLA typing. Font type represent accuracy of match - non-bold = all fields match; Bold = 2nd field mismatch; italic = 1st field mismatch

| | Right | Wrong | Total | Percent |
|---|---|---|---|---|
| 1st | 88 | 0 | 88 | 100.0% |
| 2nd | 87 | 1 | 88 | 98.9% |
| 3rd | 81 | 1 | 82 | 98.8% |
| 4th | 63 | 1 | 64 | 98.4% |

| IHW ID | Technique | C* | | DPA1* | |
|---|---|---|---|---|---|
| S20 | Reference | C*07:02:01:01 | | DPA1*01:03:01:01 | |
| | MinIon | C*07:02:01:01 | | DPA1*01:03:01:01 | |
| S27 | Reference | C*02:02:02:01 | C*08:02:01:01 | DPA1*01:03:01:01 | |
| | MinIon | C*02:02:02:01 | C*08:02:01:01 | DPA1*01:03:01:01 | |
| | Flongle | C*02:02:02:01 | C*08:02:01:01 | DPA1*01:03:01:01 | |
| IHW09377 | Reference | C*01:02:01 | C*16:01:01:01 | DPA1*01:03:01 | DPA1*02:01:01 |
| | MinIon | C*01:02:01 | C*16:01:01:01 | DPA1*01:03:01 | DPA1*02:01:01 |
| IHW01093 | Reference | C*06:02:01:01 | C*16:01:00 | DPA1*01:03:01:02 | DPA1*01:03:01:05 |
| | MinIon | C*06:02:01:01 | C*16:01:01:01 | DPA1*01:03:01:02 | DPA1*01:03:01:05 |
| IHW09381 | Reference | C*07:02:01:01 | C*05:01:01:01 | DPA1*01:03:01:01 | DPA1*01:03:01:05 |
| | MinIon | C*07:02:01:01 | C*05:01:01:01 | DPA1*01:03:01:01 | DPA1*01:03:01:05 |
| IHW01141 | Reference | C*07:01:01:01 | C*07:02:01:03 | DPA1*01:03:01:04 | DPA1*01:03:01:05 |
| | MinIon | C*07:01:01:01 | C*07:02:01:03 | DPA1*01:03:01:04 | DPA1*01:03:01:05 |
| IHW09021 | Reference | C*17:01:01:02 | | DPA1*02:02:02 | DPA1*03:01 |
| | MinIon | C*17:01:01:02 | | DPA1*02:02:02 | DPA1*03:01 |
| IHW09107 | Reference | C*01:02:01 | | DPA1*02:02:02 | DPA1*01:03:01 |
| | MinIon | C*01:02:01 | | DPA1*02:02:02 | DPA1*02:02:02 |
| IHW09388 | Reference | C*03:04:01:01 | | DPA1*01:03:01:04 | DPA1*01:03:01:05 |
| | MinIon | C*03:04:01:01 | | DPA1*01:03:01:04 | DPA1*01:03:01:05 |
| IHW01175 | Reference | C*03:03:01:01 | C*07:01:01:01 | DPA1*01:03:01:02 | DPA1*02:01:07 |
| | MinIon | C*03:03:01:01 | C*07:01:01:01 | DPA1*01:03:01:02 | DPA1*02:01:07 |
| IHW09375 | Reference | C*08:02:01:01 | C*04:01:01:01 | DPA1*01:03:01:04 | DPA1*02:02:02 |
| | MinIon | C*08:02:01:01 | C*04:01:01:01 | DPA1*01:03:01:04 | DPA1*02:02:02 |
| IHW09376 | Reference | C*02:02:02:01 | | DPA1*01:03:01:02 | DPA1*02:01:01:02 |
| | MinIon | C*02:02:02:01 | | DPA1*01:03:01:02 | DPA1*02:01:01:02 |
| IHW09056 | Reference | C*12:03:01:01 | | DPA1*02:01:01 | DPA1*01:03:01 |
| | MinIon | C*12:03:01:01 | | DPA1*02:01:01 | DPA1*01:03:01 |
| IHW09367 | Reference | C*07:02:01:01 | C*12:02:02 | DPA1*02:02:02 | |
| | MinIon | C*07:02:01:01 | C*12:02:02 | DPA1*02:02:02 | |
| IHW09373 | Reference | C*07:18 | C*08:02:01:01 | DPA1*01:03:01:02 | DPA1*01:03:01:05 |
| | MinIon | C*07:18 | C*08:02:01:01 | DPA1*01:03:01:02 | DPA1*01:03:01:05 |
| IHW09024 | Reference | C*04:01:01:01 | C*03:03:01:01 | DPA1*02:02:02 | |
| | MinIon | C*04:01:01:01 | C*03:03:01:01 | DPA1*02:02:02 | |
| IHW09045 | Reference | C*15:02:01:01 | C*07:04:01:01 | DPA1*01:03:01:05 | DPA1*01:03:01:02 |
| | MinIon | C*15:02:01:01 | C*07:04:01:01 | DPA1*01:03:01:05 | DPA1*01:03:01:02 |

| IHW ID | Technique | DPB1* | | DQA1* | |
|---|---|---|---|---|---|
| S20 | Reference | DPB1*02:01:02:01 | DPB1*03:01:01:01 | DQA1*01:02:01:01 | DQA1*03:01:01:01 |
| | MinIon | DPB1*02:01:02:01 | DPB1*03:01:01:01 | DQA1*01:02:01:01 | DQA1*03:01:01:01 |
| S27 | Reference | DPB1*01:01:01:01 | DPB1*17:01:00:01 | DQA1*03:01:01:01 | DQA1*05:01:01:01 |
| | MinIon | DPB1*01:01:01:01 | DPB1*17:01:00:01 | DQA1*03:01:01:01 | DQA1*05:01:01:01 |
| | Flongle | DPB1*01:01:01:01 | DPB1*17:01:00:01 | DQA1*03:01:01:01 | DQA1*05:01:01:01 |
| IHW09377 | Reference | DPB1*11:01:01 | DPB1*04:01:01 | DQA1*02:01 | DQA1*01:04:01:01 |
| | MinIon | DPB1*11:01:01 | DPB1*04:01:01 | DQA1*02:01 | DQA1*01:04:01:01 |
| IHW01093 | Reference | DPB1*04:01:01:01 | DPB1*04:02:01:01 | DQA1*03:01:01:01 | DQA1*05:05:01:01 |
| | MinIon | DPB1*04:01:01:01 | DPB1*04:02:01:01 | DQA1*03:01:01:01 | DQA1*05:05:01:01 |
| IHW09381 | Reference | DPB1*02:02 | DPB1*04:02:01:02 | DQA1*03:01:01 | DQA1*05:01:01:01 |
| | MinIon | DPB1*02:02 | DPB1*04:02:01:02 | DQA1*03:01:01 | DQA1*05:01:01:01 |
| IHW01141 | Reference | DPB1*04:01:01:01 | DPB1*04:02:01:02 | DQA1*05:01:01:05 | DQA1*05:01:01:02 |
| | MinIon | DPB1*04:01:01:01 | DPB1*04:02:01:02 | DQA1*05:01:01:05 | DQA1*05:01:01:02 |
| IHW09021 | Reference | DPB1*01:01:01 | DPB1*105:01 | DQA1*04:01:01:01 | DQA1*04:02 |
| | MinIon | DPB1*01:01:01 | DPB1*105:01 | DQA1*04:01:01:01 | DQA1*04:02 |
| IHW09107 | Reference | DPB1*05:01:01 | | DQA1*01:01:01:01 | DQA1*03:01:01:01 |
| | MinIon | DPB1*05:01:01 | | DQA1*01:01:01:01 | DQA1*03:01:01:01 |
| IHW09388 | Reference | DPB1*04:01:01:01 | DPB1*04:02:01:02 | DQA1*03:03:01:01 | DQA1*03:01:01 |
| | MinIon | DPB1*04:01:01:01 | DPB1*04:02:01:02 | DQA1*03:03:01:01 | DQA1*03:01:01 |
| IHW01175 | Reference | DPB1*04:01:01:01 | DPB1*13:01:01 | DQA1*03:01:01 | DQA1*01:02:01:04 |
| | MinIon | DPB1*04:01:01:01 | DPB1*13:01:01 | DQA1*03:01:01 | DQA1*01:02:01:04 |
| IHW09375 | Reference | DPB1*04:01:01:01 | DPB1*05:01:01 | DQA1*05:05:01:01 | |
| | MinIon | DPB1*04:01:01:01 | DPB1*05:01:01 | DQA1*05:05:01:01 | |
| IHW09376 | Reference | DPB1*14:01:01 | DPB1*02:01:02 | DQA1*01:02:01:01 | DQA1*02:01:01:01 |
| | MinIon | DPB1*14:01:01 | DPB1*02:01:02 | DQA1*01:02:01:01 | DQA1*02:01:01:01 |
| IHW09056 | Reference | DPB1*13:01:01 | DPB1*02:01:02 | DQA1*01:04:01:01 | DQA1*01:02:01:01 |
| | MinIon | DPB1*13:01:01 | DPB1*02:01:02 | DQA1*01:04:01:01 | DQA1*01:02:01:01 |
| IHW09367 | Reference | DPB1*05:01:01 | | DQA1*03:02 | |
| | MinIon | DPB1*05:01:01 | | DQA1*03:02 | |
| IHW09373 | Reference | DPB1*04:02:01:02 | DPB1*104:01 | DQA1*01:01:02 | DQA1*01:02:01:04 |
| | MinIon | DPB1*04:02:01:02 | DPB1*104:01 | DQA1*01:01:02 | DQA1*01:02:01:04 |
| IHW09024 | Reference | DPB1*05:01:01 | | DQA1*03:01:01 | |
| | MinIon | DPB1*05:01:01 | | DQA1*03:01:01 | |
| IHW09045 | Reference | DPB1*02:01:02 | DPB1*04:02:01 | DQA1*05:05:01:01 | DQA1*05:05:01:01 |
| | MinIon | DPB1*02:01:02 | DPB1*04:02:01 | DQA1*05:05:01:01 | DQA1*05:05:01:01 |
| IHW ID | Technique | DQB1* | | DRB1* | |
| S20 | Reference | DQB1*03:01:01:01 | DQB1*06:02:01:01 | DRB1*04:07:01:01 | DRB1*15:01:01:01 |
| | MinIon | DQB1*03:01:01:01 | DQB1*06:02:01:01 | DRB1*04:07:01:01 | DRB1*15:01:01:01 |
| S27 | Reference | DQB1*02:01:01:01 | | DRB1*03:01:01:01 | DRB1*09:01:02:01 |
| | MinIon | DQB1*02:01:01:01 | | DRB1*03:01:01:01 | DRB1*09:01:02:01 |
| | Flongle | DQB1*02:01:01:01 | | DRB1*03:01:01:01 | DRB1*09:01:02:01 |
| IHW09377 | Reference | DQB1*02:02:01 | DQB1*05:03:01 | DRB1*14:54:01 | DRB1*07:01:01 |
| | MinIon | DQB1*02:02:01 | DQB1*05:03:01 | DRB1*14:54:01 | DRB1*07:01:01 |
| IHW01093 | Reference | DQB1*03:02:01 | DQB1*03:01:01:03 | DRB1*04:01:01:01 | DRB1*11:01:01:01 |
| | MinIon | DQB1*03:02:01 | DQB1*03:01:01:03 | DRB1*04:01:01:01 | DRB1*11:01:01:01 |
| IHW09381 | Reference | DQB1*03:02:01 | DQB1*02:01:01 | DRB1*04:07:01 | DRB1*03:15:01 |
| | MinIon | DQB1*03:02:01 | DQB1*02:01:01 | DRB1*04:07:01 | DRB1*03:15:01 |
| IHW01141 | Reference | DQB1*03:01:01:01 | DQB1*02:01:01 | DRB1*12:01:01:03 | DRB1*03:01:01:01 |
| | MinIon | DQB1*03:01:01:01 | DQB1*02:01:01 | DRB1*12:01:01:03 | DRB1*03:01:01:01 |
| IHW09021 | Reference | DQB1*04:02:01 | | DRB1*03:02:01 | |
| | MinIon | DQB1*04:02:01 | | DRB1*03:03 | |
| IHW09107 | Reference | DQB1*04:01:01 | | DRB1*04:05:01 | |
| | MinIon | DQB1*04:01:01 | | DRB1*04:05:01 | |
| IHW09388 | Reference | DQB1*03:01:01:01 | DQB1*03:02:01 | DRB1*04:01:01:01 | DRB1*04:04:01 |
| | MinIon | DQB1*03:01:01:01 | DQB1*03:02:01 | DRB1*04:01:01:01 | DRB1*04:04:01 |
| IHW01175 | Reference | DQB1*03:02:01 | DQB1*06:09:01 | DRB1*04:01:01:01 | DRB1*13:02:01 |
| | MinIon | DQB1*03:02:01 | DQB1*06:09:01 | DRB1*04:01:01:01 | DRB1*13:02:01 |
| IHW09375 | Reference | DQB1*03:01:01:02 | DQB1*03:01:01:03 | DRB1*11:01:01:01 | DRB1*11:04:01 |
| | MinIon | DQB1*03:01:01:02 | DQB1*03:01:01:03 | DRB1*11:01:01:01 | DRB1*11:04:01 |
| IHW09376 | Reference | DQB1*06:02:01 | DQB1*02:02:01:01 | DRB1*11:01:02 | DRB1*07:01:01:01 |
| | MinIon | DQB1*06:02:01 | DQB1*02:02:01:01 | DRB1*11:01:02 | DRB1*07:01:01:01 |
| IHW09056 | Reference | DQB1*05:03:01 | DQB1*06:04:01 | DRB1*13:02:01 | DRB1*14:54:01 |
| | MinIon | DQB1*05:03:01 | DQB1*06:04:01 | DRB1*13:01:01 | DRB1*14:54:01 |
| IHW09367 | Reference | DQB1*03:03:02:02 | | DRB1*09:01:02 | |
| | MinIon | DQB1*03:03:02:02 | | DRB1*09:01:02 | |
| IHW09373 | Reference | DQB1*06:09:01 | DQB1*05:01:01:01 | DRB1*13:02:01 | DRB1*01:02:01 |
| | MinIon | DQB1*06:09:01 | DQB1*05:01:01:01 | DRB1*13:02:01 | DRB1*01:02:01 |
| IHW09024 | Reference | DQB1*03:02:01 | | DRB1*04:03:01 | DRB1*04:06:01 |
| | MinIon | DQB1*03:02:01 | | DRB1*04:03:01 | DRB1*04:06:01 |
| IHW09045 | Reference | DQB1*03:01:01 | | DRB1*11:04:01 | DRB1*12:01:01 |
| | MinIon | DQB1*03:01:01 | | DRB1*11:04:01 | DRB1*12:01:01 |

Field Concordance

| Fields | Right | Wrong | Total | Percent |
|---|---|---|---|---|
| 1st | 66 | 0 | 66 | 100.0% |
| 2nd | 65 | 1 | 66 | 98.5% |
| 3rd | 60 | 1 | 61 | 98.4% |
| 4th | 23 | 1 | 24 | 95.8% |

| IHW ID | Technique | DRB3* | | DRB4* | | DRB5* |
|---|---|---|---|---|---|---|
| S20 | Reference | | | DRB4*01:03:01:01 | | DRB5*01:01:01:01 |
| | MinIon | | | DRB4*01:03:01:01 | | DRB5*01:01:01:01 |
| S27 | Reference | DRB3*02:02:01:01 | | DRB4*01:01:01:01 | | |
| | MinIon | DRB3*02:02:01:01 | | DRB4*01:01:01:01 | | |
| | Flongle | DRB3*02:02:01:01 | | DRB4*01:01:01:01 | | |
| IHW09377 | Reference | DRB3*02:02:01:01 | | DRB4*01:01:02:01 | | |
| | MinIon | DRB3*02:02:01:01 | | DRB4*01:01:02:01 | | |
| IHW01093 | Reference | DRB3*02:02:01:02 | | DRB4*01:03:01:01 | | |
| | MinIon | DRB3*02:02:01:02 | | DRB4*01:03:01:01 | | |
| IHW09381 | Reference | DRB3*02:02:01:01 | | DRB4*01:03:01:01 | | |
| | MinIon | DRB3*02:02:01:01 | | DRB4*01:03:01:01 | | |
| IHW01141 | Reference | DRB3*01:01:02:01 | DRB3*02:02:01:01 | DRB4*03:01N | | |
| | MinIon | DRB3*01:01:02:01 | DRB3*02:02:01:01 | DRB4*03:01N | | |
| IHW09021 | Reference | DRB3*01:01:02:01 | | DRB4*01:01:01:01 | | |
| | MinIon | DRB3*01:01:02:01 | | DRB4*01:01:01:01 | | |
| IHW09107 | Reference | | | DRB4*01:03:01:01 | | |
| | MinIon | | | DRB4*01:03:01:01 | | |
| IHW09388 | Reference | | | DRB4*01:03:01:01 | | |
| | MinIon | | | DRB4*01:03:01:01 | | |
| IHW01175 | Reference | DRB3*03:01:01 | | DRB4*01:03:01:03 | | |
| | MinIon | DRB3*03:01:01 | | DRB4*01:03:01:03 | | |
| IHW09375 | Reference | DRB3*02:02:01:02 | | | | |
| | MinIon | DRB3*02:02:01:02 | | | | |
| IHW09376 | Reference | DRB3*02:02:02:01 | | DRB4*01:01:01:01 | | |
| | MinIon | DRB3*02:02:02:01 | | DRB4*01:01:01:01 | | |
| IHW09056 | Reference | DRB3*02:02:01:02 | | DRB4*01:01:01:01 | | |
| | MinIon | DRB3*02:02:01:02 | | DRB4*01:01:01:01 | | |
| IHW09367 | Reference | | | DRB4*01:03:01:01 | DRB4*01:03:02 | |
| | MinIon | | | DRB4*01:03:01:01 | DRB4*01:03:02 | |
| IHW09373 | Reference | DRB3*03:01:01 | | | | |
| | MinIon | DRB3*03:01:01 | | | | |
| IHW09024 | Reference | | | DRB4*01:03:01:01 | | |
| | MinIon | | | DRB4*01:03:01:01 | | |
| IHW09045 | Reference | DRB3*02:02:01:01 | | | | |
| | MinIon | DRB3*02:02:01:01 | | | | |

Field Concordance

| Fields | Right | Wrong | Total | Percent |
|---|---|---|---|---|
| 1st | 27 | | 27 | 100% |
| 2nd | 27 | | 27 | 100% |
| 3rd | 26 | | 26 | 100% |
| 4th | 23 | | 23 | 100% |

### REFERENCES

1. Linden P K. History of solid organ transplantation and organ donation. Crit Care Clin. 2009; 25(1):165-84, ix.
2. Colaneri J. An Overview of Transplant Immunosuppression—History, Principles, and Current Practices in Kidney Transplantation. Nephrol Nurs J. 2014; 41(6):549-60; quiz 61.
3. Terminology: nomenclature for factors of the HLA system, 1980. World Health Organization. Immunology. 1982; 46(1):231-4.
4. Williams T M. Human leukocyte antigen gene polymorphism and the histocompatibility laboratory. J Mol Diagn. 2001; 3(3):98-104.
5. Nunes E, Heslop H, Fernandez-Vina M, Taves C, Wagenknecht D R, Eisenbrey A B, et al. Definitions of histocompatibility typing terms. Blood. 2011; 118(23):e180-3.
6. Montgomery R A, Tatapudi V S, Leffell M S, Zachary A A. HLA in transplantation. Nat Rev Nephrol. 2018; 14(9):558-70.
7. Tiercy J M. How to select the best available related or unrelated donor of hematopoietic stem cells? Haematologica. 2016; 101(6):680-7.
8. Lazaro A, Tu B, Yang R, Xiao Y, Kariyawasam K, Ng J, et al. Human leukocyte antigen (HLA) typing by DNA sequencing. Methods Mol Biol. 2013; 1034:161-95.
9. Olerup O, Zetterquist H. HLA-DR typing by PCR amplification with sequence-specific primers (PCR-SSP) in 2 hours: an alternative to serological DR typing in clinical practice including donor-recipient matching in cadaveric transplantation. Tissue Antigens. 1992; 39(5):225-35.
10. Wang C, Krishnakumar S, Wilhelmy J, Babrzadeh F, Stepanyan L, Su L F, et al. High-throughput, high-fidelity HLA genotyping with deep sequencing. Proc Natl Acad Sci USA. 2012; 109(22):8676-81.
11. Shah N, Decker W K, Lapushin R, Xing D, Robinson S N, Yang H, et al. HLA homozygosity and haplotype bias among patients with chronic lymphocytic leukemia: implications for disease control by physiological immune surveillance. Leukemia. 2011; 25(6): 1036-9.
12. Levene M J, Korlach J, Turner S W, Foquet M, Craighead H G, Webb W W. Zero-mode waveguides for single-molecule analysis at high concentrations. Science. 2003; 299(5607):682-6.
13. Stoddart D, Heron A J, Mikhailova E, Maglia G, Bayley H. Single-nucleotide discrimination in immobilized DNA oligonucleotides with a biological nanopore. Proc Natl Acad Sci USA. 2009; 106(19):7702-7.
14. Delaneau O, Howie B, Cox A J, Zagury J F, Marchini J. Haplotype estimation using sequencing reads. Am J Hum Genet. 2013; 93(4):687-96.
15. Dilthey A, Cox C, Iqbal Z, Nelson M R, McVean G. Improved genome inference in the MHC using a population reference graph. Nat Genet. 2015; 47(6):682-8.
16. Petersdorf E W, Malkki M, O'HUigin C, Carrington M, Gooley T, Haagenson M D, et al. High HLA-D P Expression and Graft-versus-Host Disease. N Engl J Med. 2015; 373(7):599-609.
17. De Coster W, D'Hert S, Schultz D T, Cruts M, Van Broeckhoven C. NanoPack: visualizing and processing long-read sequencing data. Bioinformatics. 2018; 34(15):2666-9.
18. Li H. Minimap2: pairwise alignment for nucleotide sequences. Bioinformatics. 2018; 34(18):3094-100.
19. Li H. A statistical framework for SNP calling, mutation discovery, association mapping and population genetical parameter estimation from sequencing data. Bioinformatics. 2011; 27(21):2987-93.
20. Li H, Handsaker B, Wysoker A, Fennell T, Ruan J, Homer N, et al. The Sequence Alignment/Map format and SAMtools. Bioinformatics. 2009; 25(16):2078-9.
21. Dilthey A T, Gourraud P A, Mentzer A J, Cereb N, Iqbal Z, McVean G. High-Accuracy HLA Type Inference from Whole-Genome Sequencing Data Using Population Reference Graphs. PLoS Comput Biol. 2016; 12(10):e1005151.
22. Garrison E, G. M. Haplotype-based variant detection from short-read sequencing. arXiv preprint. 2012; arXiv:1207.3907 [q-bio.GN].
23. Patterson M, Marschall T, Pisanti N, van lersel L, Stougie L, Klau G W, et al. WhatsHap: Weighted Haplotype Assembly for Future-Generation Sequencing Reads. J Comput Biol. 2015; 22(6):498-509.
24. Bunce M, Passey B. HLA typing by sequence-specific primers. Methods Mol Biol. 2013; 1034:147-59.
25. Yin Y, Lan J H, Nguyen D, Valenzuela N, Takemura P, Bolon Y T, et al. Application of High-Throughput Next-Generation Sequencing for HLA Typing on Buccal Extracted DNA: Results from over 10,000 Donor Recruitment Samples. PLoS One. 2016; 11(10):e0165810.
26. Jia H, Guo Y, Zhao W, Wang K. Long-range PCR in next-generation sequencing: comparison of six enzymes and evaluation on the MiSeq sequencer. Sci Rep. 2014; 4:5737.
27. Castelli E C, Mendes-Junior C T, Veiga-Castelli L C, Pereira N F, Petzl-Erler M L, Donadi E A. Evaluation of computational methods for the reconstruction of HLA haplotypes. Tissue Antigens. 2010; 76(6):459-66.
28. Lee P L. DNA amplification in the field: move over PCR, here comes LAMP. Mol Ecol Resour. 2017; 17(2):138-41.
29. Gabrieli T, Sharim H, Fridman D, Arbib N, Michaeli Y, Ebenstein Y. Selective nanopore sequencing of human BRCA1 by Cas9-assisted targeting of chromosome segments (CATCH). Nucleic Acids Res. 2018; 46(14):e87.
30. Watson C M, Crinnion L A, Hewitt S, Bates J, Robinson R, Carr I M, et al. Cas9-based enrichment and single-molecule sequencing for precise characterization of genomic duplications. Lab Invest. 2019.
31. Liu Q, Fang L, Yu G, Wang D, Xiao C L, Wang K. Detection of DNA base modifications by deep recurrent neural network on Oxford Nanopore sequencing data. Nat Commun. 2019; 10(1):2449.
32. Soneson C, Yao Y, Bratus-Neuenschwander A, Patrignani A, Robinson M D, Hussain S. A comprehensive examination of Nanopore native RNA sequencing for characterization of complex transcriptomes. Nat Commun. 2019; 10(1):3359.
33. Jain M, Koren S, Miga K H, Quick J, Rand A C, Sasani T A, et al. Nanopore sequencing and assembly of a human genome with ultra-long reads. Nat Biotechnol. 2018; 36(4):338-45.
34. Bertaina A, Andreani M. Major Histocompatibility Complex and Hematopoietic Stem Cell Transplantation: Beyond the Classical HLA Polymorphism. Int J Mol Sci. 2018; 19(2).
35. Park M, Seo J J. Role of HLA in Hematopoietic Stem Cell Transplantation. Bone Marrow Res. 2012; 2012:680841.
36. Liu C, Xiao F, Hoisington-Lopez J, Lang K, Quenzel P, Duffy B, et al. Accurate Typing of Human Leukocyte Antigen Class I Genes by Oxford Nanopore Sequencing. J Mol Diagn. 2018; 20(4):428-35.
37. Shiina T, Suzuki S, Ozaki Y, Taira H, Kikkawa E, Shigenari A, et al. Super high resolution for single molecule-sequence-based typing of classical HLA loci at the 8-digit level using next generation sequencers. Tissue Antigens. 2012; 80(4):305-16.
38. Juhos S., Rigo K., Horvath G., On Genotyping Polymorphic HLA Genes—Ambiguities and Quality Measures Using NGS. Next Generation Sequencing—Advances, Applications and Challenges 2016, 13:369-386. DOI: 10.5772/61592.

1. A set of oligonucleotides comprising oligonucleotides of SEQ ID NOs: 1-11, 16-35 and 37-42 or variants thereof.

2. The set of oligonucleotides according to claim 1, further comprising one or more of oligonucleotides of SEQ ID NOs: 12, 13, 14, 15 and 36 or variants thereof.

3. A kit compromising the set of oligonucleotides according to claim 1 or claim 2.

4. The kit according to claim 3, further comprising one or more of a set of instructions, a DNA amplification mix, nuclease free water, a barcoding mix, a ligation mix, an end repairing mix, a tailing mix, a clean-up mix, an adaptor mix, and an elution buffer.

5. The kit according to claim 3 or claim 4, wherein the DNA amplification mix comprises a DNA polymerase and dNTPs.

6. The kit according to claim 5, the DNA polymerase is a Taq polymerase.

7. The kit according to any of claims 4-6, comprising a DNA polymerase with 3′ to 5′ exonuclease activity.

8. The set of oligonucleotides according to claim 2, or the kit according to any of claims 3-7, wherein the oligonucleotides of SEQ ID NOs: 1-11, 16-35 and 37-42 or variants thereof are provided separately from the one or more of the oligonucleotides of SEQ ID NOs: 12, 13, 14, 15 and 36 or variants thereof; or
wherein the oligonucleotides of SEQ ID NOs: 1-11, 16-35 and 37-42 or variants thereof are provided together with the one or more of the oligonucleotides of SEQ ID NOs: 12, 13, 14, 15 and 36 or variants thereof.

9. The set of oligonucleotides according to claim 1, 2 or 8 or the kit according to any of claims 3-8, wherein the oligonucleotides are be provided lyophilised or in a suitable buffer.

10. The set of oligonucleotides according to claim 1, 2, 8 or 9, or the kit according to any of claims 3-9, for use in determining the HLA genotype of a DNA sample.

11. A method of determining the HLA genotype of a DNA sample comprising
a) contacting the oligonucleotides or variants thereof according to any of claim 1-2 or 8-10, with the DNA sample and a DNA amplification mix,
the DNA amplification mix optionally comprising one or more of a DNA polymerase such as a Taq polymerase, a DNA polymerase with 3′ to 5′ exonuclease activity and dNTPs and;
b) amplifying target sequences in the DNA sample using a primer-dependent DNA amplification method, such as PCR, thereby producing amplicons; and
c) determining the sequence of said amplicons.

12. The method of claim 12, wherein step a) and step b) is performed independently for oligonucleotides of SEQ ID NOs: 1-11, 16-35 and 37-42 or variants thereof, and for the one or more oligonucleotides of SEQ ID NOs: 12, 13, 14, 15 and 36 or variants thereof.

13. The method of claim 12, wherein the amplification products are combined for step c).

14. The method of any of claims 11-13, wherein the oligonucleotides of SEQ ID NO: 1-6 are provided for use at a concentration of about 20-200 μM, about 50-150 μM, such as about 100 μM per 25 μL amplification reaction in step b).

15. The method of any of claims 11-14, wherein the oligonucleotides of SEQ ID NO: 7-42 are provided for use at a concentration of about 5-100 μM, about 10-50 μM, such as about 20 μM per 25 μL amplification reaction in step b).

16. The method of any of claims 11-15, wherein the DNA sample is a sample of DNA from a human subject, optionally wherein the DNA has been extracted from a blood or tissue sample obtained from the subject.

17. The method of any of claims 11-16, wherein the amplification method in step b) comprises or consists of the use of a thermocycling profile comprising or consisting of the cycling conditions:
i) about 95° C. for about 2 minutes;
ii) about 30 cycles, such as between 20 and 40 cycles, of: about 94° C. for about 30 seconds and about 65° C. for between about 4 and about 10 minutes, such as 4 minutes, 5 minutes, 6 minutes, 7 minutes, 8 minutes, 9 minutes, or 10 minutes; and
iii) a final extension at about 72° C. for about 10 minutes.

18. The method of any of claims 11-17, wherein all DNA amplification reactions are performed in the same thermocycler, or wherein each amplification reaction is performed independently.

19. The method of any of claims 11-18, wherein the method further comprises one or more of the steps of end repairing of the amplicons, adding a molecular barcode ‘tail’ to the amplicon, ‘clean-up’ of the amplicons, sorting the amplicons by size, and amplicon quantification.

20. The method of any of claims 11-19, wherein in step c) of the method, the sequences of amplicons may be determined using a next generation sequencing (NGS) method, for example Oxford Nanopore® Technology.

21. The method of any of claims 11-20, further comprising comparing the determined sequences of the amplicons with the DNA sequences of known HLA types.

22. The method of any of claims 11-21, further comprising haplotype phasing, and/or identification of homozygosity.

23. The method of any of claims 11-22, for use in identifying a suitable donor and/or recipient of a transplant, paternity testing, identifying the HLA type for determination of epitope binding capability in neo-antigen prediction, or diagnosing an immune disorder such as ankylosing spondylitis.

24. The method of identifying a suitable donor and/or recipient of a transplant according to claim 23, wherein the transplant is a kidney transplant, heart transplant, bone marrow transplant, stem cell transplant, liver transplant, lung transplant, pancreas transplant, small bowel transplant, or uterine transplant.

25. The method of any of claims 11-24, further comprising the step
d) identifying a suitable transplant donor and/or recipient when there is at least a one field match between donor and recipient, and optionally wherein in step d) there is a two field, three field or four field match between donor and recipient.
